(19) United States
(12) Patent Application Publication
(10) Pub. No.: US 2005/0186582 A1
(43) Pub. Date: Aug. 25, 2005

(54) Compositions and methods of using hexokinase V

(75) Inventors: Jeffrey D. Johnson, Moraga, CA (US); Francine M. Gregoire, Lafayette, CA (US); Anthony Schweitzer, Redwood City, CA (US); Yuko Terasawa, Campbell, CA (US); Maria E. Wilson, San Francisco, CA (US); John E. Blume, Danville, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: METABOLEX, INC., Hayward, CA (US)

(21) Appl. No.: 10/805,075

(22) Filed: Mar. 19, 2004

Related U.S. Application Data
(60) Provisional application No. 60/456,650, filed on Mar. 20, 2003.

(51) Int. Cl. C12Q 1/68; A61K 48/00

(52) U.S. Cl. 435/6; 514/44

(57) ABSTRACT

The current invention provides methods of identifying modulators of hexokinase V. Such modulators can be used for the treatment of diabetes or for delaying the onset of diabetes. The invention also provides methods of diagnosing diabetes or pre-diabetes and methods of making a prognosis based on the detection of hexokinase V nucleic acids and proteins.

### CROSS-REFERENCE TO RELATED APPLICATIONS

This application claims the benefit of U.S. Provisional Application No. 60/456,650, filed Mar. 20, 2003, which application is herein incorporated by reference.

### BACKGROUND OF THE INVENTION

Diabetes mellitus can be divided into two clinical syndromes, Type 1 and Type 2. Type 1, or insulin-dependent diabetes mellitus (IDDM), is a chronic autoimmune disease characterized by the extensive loss of beta cells in the pancreatic Islets of Langerhans, which produce insulin. As these cells are progressively destroyed, the amount of secreted insulin decreases, eventually leading to hyperglycemia (abnormally high level of glucose in the blood) when the amount of secreted insulin drops below the level required for euglycemia (normal blood glucose level). Although the exact trigger for this immune response is not known, patients with IDDM have high levels of antibodies against proteins expressed in pancreatic beta cells. However, not all patients with high levels of these antibodies develop IDDM.

Type 2 diabetes (also referred to as non-insulin dependent diabetes mellitus (NIDDM)) develops when muscle, fat and liver cells fail to respond normally to insulin. This failure to respond (insulin resistance) may be due to reduced numbers of insulin receptors on these cells, or a dysfunction of signaling pathways within the cells, or both. The beta cells initially compensate for insulin resistance by increasing insulin output. Over time, however, the beta cells become unable to produce enough insulin to maintain normal glucose levels, indicating progression to Type 2 diabetes. This beta cell insufficiency may arise from a decline in total beta cell mass or from a decline in the ability of individual beta cells to respond appropriately to increased blood glucose.

Glucose-stimulated insulin secretion (GSIS) is mediated by a variety of metabolic and signaling components of the beta cell, but the central mechanism is related to the rate at which glucose is oxidized with the concomitant generation of ATP. Higher ATP/ADP ratios result in the closure of the K_{ATP} channel composed of Kir6.2 and SUR1 leading to plasma membrane depolarization and activation of voltage-sensitive Ca⁺⁺ channels. The resulting rise in intracellular Ca⁺⁺ is the major trigger for insulin release.

To initiate the primary stimulus to insulin release, glucose enters pancreatic beta cells by facilitated diffusion and is converted to glucose-6-phosphate by hexokinase IV, also known as glucokinase (GK). GK is the rate-limiting enzyme for glucose metabolism in the beta cell and is therefore often called the glucose sensor (Matschinsky, Diabetes 51 Suppl 3: S394-404, 2002). GK is apparently only found in the handful of cell types that respond to changes in blood glucose; the pancreatic beta cell and the liver parenchymal cells each have a unique GK isoform produced by alternative promoter usage. A few cell types in the brain and in the gut also express GK.

A variety of human mutations in GK that result in impaired GSIS have been observed. Inactivating mutations of GK lead to maturity onset diabetes of the young 2 (MODY2) if one allele is affected (Froguel et al., N Engl J Med 328:697-702, 1993) and permanent neonatal diabetes if both alleles encode inactive enzyme (Njolstad et al.,. N Engl J Med 344:1588-1592, 2001). Activating mutations of GK, which result in a decrease in the Km for glucose, cause a persistent hyperinsulinemic hypoglycemia (PHHI) (Glaser et al., N Engl J Med 338:226-230, 1998; Christensen et al., Diabetes 51:1240-1246, 2002).

Other glucose phosphorylating enzymes, hexokinases I, II and III have much lower Kms for glucose, but these enzymes are much less abundant than GK in normal beta cells (Shuit et al., J Biol Chem 274:32803-32809, 1999). Lack of low Km hexokinase expression appears to be important for the ability of GK to function as a glucose sensor; low Km hexokinases expressed in islets tend to make insulin secretion more constitutive and less responsive to changes in blood glucose levels. For example, artificial over-expression of hexokinase I in rat pancreatic islets in vitro causes a high level of insulin secretion at low glucose concentrations (Becker et al., J Biol Chem 269:21234-21238, 1994). Further, a rise in low Km hexokinase activity has been observed in rodent models of chronic beta cell stress. Milburn et al. (J Biol Chem 270:1295-1299, 1995) also observed a threefold increase in low Km hexokinase activity in the islets of obese Zucker fatty rats versus lean controls and a concomitant increase in insulin secretion and glucose utilization at low glucose (<3 mM) concentrations. Hosokawa et al., (Diabetes 44:1328-1333, 1995) observed a 2-fold increase in low Km hexokinase activity in islets remaining after 90% pancreatectomy. However, a role of hexokinases in diabetes or pre-diabetes, in particular hexokinases other than GK that are expressed in the pancreas, has not been determined.

Another hexokinase, hexokinase V (also referred to herein as HKV), has recently been identified see, e.g., WO 01/90325. The current invention is based on the discovery that hexokinase V plays a role in glucose-stimulated insulin secretion and insulin sensitivity, and in diseases that relate to glucose metabolism, e.g., diabetes.

### BRIEF SUMMARY OF THE INVENTION

The inventors have determined that overexpression of hexokinase V plays a role in disorders relating to glucose metabolism, e.g., diabetes. Thus, this invention provides methods of identifying inhibitors of HK V activity and/or expression. Such inhibitors can be used, e.g., for the treatment of disorders relating to glucose and insulin metabolism, e.g, diabetes or pre-diabetes.

Thus, in one aspect the invention provides a method for identifying an agent for treating a diabetic or pre-diabetic individual, the method comprising the steps of: (i) contacting a candidate agent, e.g., a small organic molecule, an siRNA, or an antisense RNA, with a kidney or pancreatic cell that expresses a nucleic acid encoding a polypeptide having glucose phosphorylating activity that comprises at least 20 contiguous amino acids of SEQ ID NO:2; determining the level of the polypeptide; and selecting an agent that inhibits the activity of the polypeptide, thereby identifying an agent for treating a diabetic or pre-diabetic individual. Often, the polypeptide comprises SEQ ID NO:2.

In one embodiment, the polypeptide is overexpressed in the cell relative to normal. In another embodiment, the pancreatic cell is from a diabetic animal.

The method of claim 1, wherein the step of determining the level of the polypeptide comprises determining the activity of the polypeptide.

The step of determining the level of the polypeptide often comprises determining the amount of protein present using an immunoassay.

In another aspect, the invention provides a method for identifying an agent for treating a diabetic or pre-diabetic individual, the method comprising the steps of: (i) contacting a candidate agent, e.g., an siRNA or an antisense RNA, with a kidney or pancreatic cell, e.g., a pancreatic islet cell, that overexpresses a nucleic acid encoding a polypeptide having glucose phosphorylating activity that comprises at least 20 contiguous amino acids of SEQ ID NO:2; (ii) determining the level of an RNA that encodes the polypeptide; and (ii) selecting an agent that inhibits the activity of the polypeptide, thereby identifying an agent for treating a diabetic or pre-diabetic individual. Typically, the polypeptide has an amino acid sequence as set forth in SEQ ID NO:2. In one embodiment, the pancreatic cell is from a diabetic animal.

In another embodiment, the methods of the invention can further comprise administering the agent to a diabetic or pre-diabetic animal; determining the sensitivity of insulin secretion to glucose levels in the animal; and selecting a candidate agent that improves the sensitivity of insulin secretion in response to glucose. The step of determining the sensitivity of the animal may comprises, e.g., determining the amount of insulin released from pancreatic tissue in response to glucose.

The methods of the invention can further comprise administering the agent to an animal that is a diabetic or pre-diabetic model; determining the level of the polypeptide or the nucleic acid encoding the polypeptide in a pancreatic sample from the animal; and selecting the candidate agent that decreases the level of the polypeptide or the nucleic acid.

The invention also provides a method for identifying an agent for treating a diabetic or pre-diabetic individual, the method comprising the steps of: (i) contacting a candidate agent with a polypeptide having glucose phosphorylating activity that comprises at least 20 contiguous amino acids of SEQ ID NO:2; (ii) determining the activity of the polypeptide; (iii) selecting an agent that inhibits the activity; (iv) administering the agent to a diabetic or pre-diabetic animal; (v) determining the glucose sensitivity of the animal; and (vi) selecting an agent that improves the sensitivity of the animal to insulin secretion.

In another aspect, the invention provides a method of regulating insulin release in response to glucose in a diabetic animal or a pre-diabetic animal, the method comprising administering to the animal a therapeutically effective amount of an agent identified by any of the methods described herein. Often, the animal is a human. In some embodiments, the agent is administered to pancreatic tissue.

The invention also provides a method of introducing an expression cassette into a pancreatic cell, the method comprising introducing into the cell an expression vector comprising a nucleic acid that, when expressed, inhibits the expression of a nucleic acid encoding a polypeptide having glucose phosphorylating activity that comprises at least 20 contiguous amino acids of SEQ ID NO:2. Typically, the polypeptide comprises SEQ ID NO:2. In some embodiments, the method further comprising introducing the cell into a diabetic animal. Often, the diabetic animal is a human and the cell is from a human.

In other aspects, the invention provides a method of diagnosing a prediabetic or diabetic patient; the method comprising: detecting an increase, relative to normal, in the level of a polypeptide of SEQ ID NO:2 in a sample, typically a blood or serum sample, from the patient, thereby diagnosing the diabetic or prediabetic patient.

The invention also provides an isolated nucleic acid encoding a polypeptide having an amino acid sequence as set forth in SEQ ID NO:4. In one embodiment, the isolated nucleic acid comprises the nucleic acid sequence of SEQ ID NO:3.

### BRIEF DESCRIPTION OF THE DRAWINGS

FIG. 1 shows the expression of hexokinase V mRNA in four non-diabetic human islet RNA samples and on a Type II diabetic islet sample in comparison to ten other tissue samples. Custom human islet endocrine cell oligonucleotide arrays were hybridized with cRNAs made from these sample and analyzed after global scaling of the chip data. The Average Difference Score is a measure of the abundance of the specific mRNA in each tissue. The relative abundance of mRNA for cyclophilin is also shown for comparison.

FIGS. 2a-2d show the sequence comparison of human hexokinase V to other hexokinases.

FIG. 3 shows the results of a hybridization of a hexokinase V probe to a multiple tissue northern blot of human mRNAs.

FIG. 4 shows an RT-PCR analysis of hexokinase expression in human tissues. HK, hexokinase.

FIG. 5 shows the results of an immunofluorescence analysis of hexokinase V expression in human pancreatic tissue. Hexokinase V is co-expressed with insulin in human tissue.

FIGS. 6a and 6b show the analysis of recombinant human hexokinase V that was expressed and purified from E. coli. FIG. 6a shows electrophoretic analysis of the recombinant protein; FIG. 6b shows the glucose phosphorylating of recombinant HKV (squares) and HK I (circles). A commercial preparation of bovine HK I (triangles) is shown for comparison. About 1 mg of purified protein was used for each determination.

FIG. 7 shows a graph depicting the glucose Km of recombinant hexokinase V. The Km of glucose for HK V was calculated to be 0.15 mM in SOFTmaxPro software in 4-parameter graph type. The hexokinase activity assay was measured on the FlexStation with final D-glucose concentrations ranging from 0-30 mM in the reaction mixture with approximately 1 μg of purified HK V protein (eluted without glucose).

FIG. 8 shows hexokinase V mRNA levels evaluated by real-time PCR in islets derived from 12 week-old lean (db/+) and diabetic (db/db) mice and in islets derived from C57BL/6J mice fed a chow or high fat (58% fat) diet for 16 weeks.

FIG. 9 shows immunohistochemical detection of mouse hexokinase V in non-diabetic and diabetic mouse tissues.

FIG. 10 shows the results of overexpreesion of HKV in adenoviral infected islet cells.

### DETAILED DESCRIPTION OF THE INVENTION

Definitions

“Insulin sensitivity” refers to the ability of a cell or tissue to respond to insulin. Responses include, e.g., glucose uptake of a cell or tissue in response to insulin stimulation. Sensitivity can be determined at an organismal, tissue or cellular level. For example, blood or urine glucose levels following a glucose tolerance test are indicative of insulin sensitivity. Other methods of measuring insulin sensitivity include, e.g., measuring glucose uptake (see, e.g., Garcia de Herreros, A., and Birnbaum, M. J. J. Biol. Chem. 264, 19994-19999 (1989); Klip, A., Li, G., and Logan, W. J. Am. J. Physiol. 247, E291-296 (1984)), measuring the glucose infusion rate (GINF) into tissue such as the skeletal muscle (see, e.g., Ludvik et al., J. Clin. Invest. 100:2354 (1997); Frias et al., Diabetes Care 23:64, (2000)) and measuring sensitivity of GLUT4 translocation (e.g., as described herein) in response to insulin.

The “response to glucose” as used herein refers to the ability of an animal to respond to levels of glucose in the serum. This can be measured using various assays well known to those in the art. Such assays include, are not limited to, analysis of fasting blood glucose levels, analysis of fasting insulin levels, assessment of glucose levels during an oral or intraperitoneal glucose tolerance test, assessment of insulin or C-peptide levels during an oral or intraperitoneal glucose tolerance. Additionally, other secretagogues, e.g., arginine or glyburide, can be used to test for glucose specificity of the improvement in islet function.

Typically, a response to blood glucose levels is measured by assessing the level of insulin secretion. The term “sensitivity of glucose-stimulated insulin secretion” or “sensitivity of insulin secretion to glucose levels” refers to the ability of pancreatic cells to release insulin in response to glucose levels. “Insensitive” in this context typically means that insulin secretion in response to normal, i.e., about 5.6 mM, or higher glucose levels is reduced in comparison to insulin secretion in normal, non-diabetic (lean) pancreatic cells.

“Activity” of an HKV polypeptide refers to structural, regulatory, or biochemical functions of the polypeptide in its native cell or tissue. Activity of HKV include both direct activities and indirect activities. An exemplary direct activity is glucose phosphorylation or phosphorylation of other hexoses. Exemplary indirect activities are observed as a change in phenotype or response in a cell or tissue to a polypeptide's direct activity, e.g., modulating the sensitivity of insulin secretion to glucose levels or modulation of insulin sensitivity of a cell as a result of the interaction of the polypeptide with other cellular or tissue components.

“Predisposition for diabetes” occurs in a person when the person is at high risk for developing diabetes. A number of risk factors are known to those of skill in the art and include: genetic factors (e.g., carrying alleles that result in a higher occurrence of diabetes than in the average population or having parents or siblings with diabetes); overweight (e.g., body mass index (BMI) greater or equal to 25 kg/m²); habitual physical inactivity, race/ethnicity (e.g., African-American, Hispanic-American, Native Americans, Asian-Americans, Pacific Islanders); previously identified impaired fasting glucose or impaired glucose tolerance, hypertension (e.g., greater or equal to 140/90 mmHg in adults); HDL cholesterol less than or equal to 35 mg/dl; triglyceride levels greater or equal to 250 mg/dl; a history of gestational diabetes or delivery of a baby over nine pounds; and/or polycystic ovary syndrome. See, e.g., “Report of the Expert Committee on the Diagnosis and Classification of Diabetes Mellitus” and “Screening for Diabetes” Diabetes Care 25(1): S5-S24 (2002).

A “non-diabetic individual” (also referred to herein as a “lean” individual), when used to compare with a sample from a patient, refers to an adult with a fasting blood glucose level less than 110 mg/dl or a 2 hour PG reading of 140 mg/dl. “Fasting” refers to no caloric intake for at least 8 hours. A “2 hour PG” refers to the level of blood glucose after challenging a patient to a glucose load containing the equivalent of 75 g anhydrous glucose dissolved in water. The overall test is generally referred to as an oral glucose tolerance test (OGTT). See, e.g., Diabetes Care, Supplement 2002, American Diabetes Association: Clinical Practice Recommendations 2002. The level of a polypeptide in a non-diabetic individual can be a reading from a single individual, but is typically a statistically relevant average from a group of non-diabetic individuals. The level of a polypeptide in a nondiabetic individual can be represented by a value, for example in a computer program.

A “pre-diabetic individual,” when used to compare with a sample from a patient, refers to an adult with a fasting blood glucose level greater than 110 mg/dl but less than 126 mg/dl or a 2 hour PG reading of greater than 140 mg/dl but less than 200 mg/dl. A “diabetic individual,” when used to compare with a sample from a patient, refers to an adult with a fasting blood glucose level greater than 126 mg/dl or a 2 hour PG reading of greater than 200 mg/dl.

A hexokinase V nucleic acid or polypeptide refers to polymorphic variants, alleles, mutants, and interspecies homologs and hexokinase domains thereof that: (1) have an amino acid sequence that has greater than about 65% amino acid sequence identity, 70%, 75%, 80%, 85%, 90%, preferably 91%, 92%, 93%, 94%, 95%, 96%, 97%, 98% or 99% or greater amino acid sequence identity, preferably over a window of at least about 25, 50, 100, 200, 500, 1000, or more amino acids, to a sequence of SEQ ID NO:2; (2) bind to antibodies raised against an immunogen comprising an amino acid sequence of SEQ ID NO:2 and conservatively modified variants thereof; (3) have at least 15 contiguous amino acids, more often, at least 20, 30, 40, 50 or 100 contiguous amino acids, of SEQ ID NO:2; (4) specifically hybridize (with a size of at least about 100, preferably at least about 500 or 1000 nucleotides) under stringent hybridization conditions to a sequence of SEQ ID NO:1 and conservatively modified variants thereof; (5) have a nucleic acid sequence that has greater than about 95%, preferably greater than about 96%, 97%, 98%, 99%, or higher nucleotide sequence identity, preferably over a region of at least about 50, 100, 200, 500, 1000, or more nucleotides, to SEQ ID NO:1; or (6) are amplified by primers that specifically hybridize under stringent conditions to SEQ ID NO:1. This term also refers to a domain of a hexokinase V or a fusion protein comprising a domain of a hexokinase V linked to a heterologous protein. A hexokinase V polynucleotide or polypeptide sequence of the invention is typically from a mammal including, but not limited to, human, mouse, rat, hamster, cow, pig, horse, sheep, or any mammal. A “hexokinase V polynucleotide” and a “hexokinase V polypeptide,” are both either naturally occurring or recombinant.

An “agonist” or “activator” refers to an agent that binds to, stimulates, increases, activates, facilitates, enhances activation, sensitizes or up regulates the activity or expression of a polypeptide of the invention.

An “antagonist” or “inhibitor” refers to an agent that binds to, partially or totally blocks stimulation, decreases, prevents, delays activation, inactivates, desensitizes, or down regulates the activity or expression of a polypeptide of the invention.

“Antibody” refers to a polypeptide substantially encoded by an immunoglobulin gene or immunoglobulin genes, or fragments thereof which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see, Paul (Ed.) Fundamental Immunology, Third Edition, Raven Press, NY (1993)). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies (e.g., single chain Fv).

The terms “peptidomimetic” and “mimetic” refer to a synthetic chemical compound that has substantially the same structural and functional characteristics of the antagonists or agonists of the invention. Peptide analogs are commonly used in the pharmaceutical industry as non-peptide drugs with properties analogous to those of the template peptide. These types of non-peptide compound are termed “peptide mimetics” or “peptidomimetics” (Fauchere, J. Adv. Drug Res. 15:29 (1986); Veber and Freidinger TINS p. 392 (1985); and Evans et al. J. Med. Chem. 30:1229 (1987), which are incorporated herein by reference). Peptide mimetics that are structurally similar to therapeutically useful peptides may be used to produce an equivalent or enhanced therapeutic or prophylactic effect. Generally, peptidomimetics are structurally similar to a paradigm polypeptide (i.e., a polypeptide that has a biological or pharmacological activity), such as apolypeptide exemplified in this application, but have one or more peptide linkages optionally replaced by a linkage selected from the group consisting of, e.g., —CH2NH—, —CH2S—, —CH2-CH2-, —CH═CH— (cis and trans), —COCH2-, —CH(OH)CH2-, and —CH2SO—. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. For example, a mimetic composition is within the scope of the invention if it is capable of carrying out the binding or other activities of an agonist or antagonist of a polypeptide of the invention.

The term “gene” means the segment of DNA involved in producing a polypeptide chain; it includes regions preceding and following the coding region (leader and trailer) as well as intervening sequences (introns) between individual coding segments (exons).

The term “isolated,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “nucleic acid” or “polynucleotide” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)). The term nucleic acid is used interchangeably with gene, cDNA, and mRNA encoded by a gene.

“siRNA” refers to small interfering RNAs, that can cause post-transcriptional silencing of specific genes in cells, for example, mammalian cells (including human cells) and in the body, for example, mammalian bodies (including humans). The phenomenon of RNA interference is described and discussed in Bass, Nature 411: 428-29 (2001); Elbahir et al., Nature 411: 494-98 (2001); and Fire et al., Nature 391: 806-11 (1998); and WO 01/75164, where methods of making interfering RNA also are discussed. The siRNAs based upon the sequences and nucleic acids encoding the gene products disclosed herein typically have fewer than 100 base pairs and can be, e.g., about 30 bps or shorter, and can be made by approaches known in the art, including the use of complementary DNA strands or synthetic approaches. Exemplary siRNAs according to the invention can have up to 29 bps, 25 bps, 22 bps, 21 bps, 20 bps, 15 bps, 10 bps, 5 bps or any integer thereabout or therebetween. Tools for designing optimal inhibitory siRNAs include that available from DNAengine Inc. (Seattle, Wash.) and Ambion, Inc. (Austin, Tex.).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but which functions in a manner similar to a naturally occurring amino acid.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins (1984)).

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (e.g., a polypeptide of the invention), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences are substantially identical if two sequences have a specified percentage of amino acid residues or nucleotides that are the same (i.e., 60% identity, optionally 65%, 70%, 75%, 80%, 85%, 90%, or 95% identity over a specified region, or, when not specified, over the entire sequence), when compared and aligned for maximum correspondence over a comparison window, or designated region as measured using one of the following sequence comparison algorithms or by manual alignment and visual inspection. The invention provides polypeptides or polynucleotides that are substantially identical to the polynucleotides or polypeptides, respectively, exemplified herein in SEQ ID NOs:1 and 2. This definition also refers to the complement of a test sequence. Optionally, the identity exists over a region that is at least about 50 nucleotides in length, or more preferably over a region that is 100 to 500 or 1000 or more nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Two examples of algorithms that are suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or 5×SSC, 1% SDS, incubating at 65° C., with wash in 0.2×SSC, and 0.1% SDS at 55° C., 60° C., or 65° C. Such washes can be performed for 5, 15, 30, 60, 120, or more minutes.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. Such washes can be performed for 5, 15, 30, 60, 120, or more minutes. A positive hybridization is at least twice background. Those of ordinary skill will readily recognize that alternative hybridization and wash conditions can be utilized to provide conditions of similar stringency.

The phrase “a nucleic acid sequence encoding” refers to a nucleic acid which contains sequence information for a structural RNA such as rRNA, a tRNA, or the primary amino acid sequence of a specific protein or peptide, or a binding site for a trans-acting regulatory agent. This phrase specifically encompasses degenerate codons (i.e., different codons which encode a single amino acid) of the native sequence or sequences that may be introduced to conform with codon preference in a specific host cell.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under-expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The phrase “specifically (or selectively) binds to an antibody” or “specifically (or selectively) immunoreactive with”, when referring to a protein or peptide, refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein. For example, antibodies raised against a protein having an amino acid sequence encoded by any of the polynucleotides of the invention can be selected to obtain antibodies specifically immunoreactive with that protein and not with other proteins, except for polymorphic variants. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein. See, Harlow and Lane Antibodies, A Laboratory Manual, Cold Spring Harbor Publications, NY (1988) for a description of immunoassay formats and conditions that can be used to determine specific immunoreactivity. Typically, a specific or selective reaction will be at least twice the background signal or noise and more typically more than 10 to 100 times background.

“Inhibitors” or “modulators” of expression or of activity are used to refer to inhibitory molecules that decrease hexokinase activity or expression. Such modulators are identified using in vitro and in vivo assays for expression or activity. Modulators encompass e.g., antagonists, and their homologs and mimetics. Inhibitors are agents that, e.g., inhibit expression of hexokinase V or bind to, partially or totally block stimulation, decrease, prevent, delay activation, inactivate, desensitize, or down regulate the activity of hexokinase V. Modulators include naturally occurring and synthetic ligands, antagonists, small chemical molecules and the like. Assays for inhibitors, e.g., applying putative modulator compounds to cells expressing hexokinase V and then determining the functional effects on activity, as described above. Samples or assays comprising a hexokinase V polypeptide that are treated with a potential modulator are compared to control samples without the modulator to examine the extent of effect. Control samples (untreated with modulators) are assigned a relative activity value of 100%. Inhibition of a polypeptide of the invention is achieved when the polypeptide activity value relative to the control is about 80%, optionally 50% or 25, 10%, 5% or 1%.

Introduction

This invention is based on the discovery that overexpression of hexokinase V plays a role in metabolic diseases such as diabetes. HKV is expressed in pancreatic cells and kidney cells. Further, HKV mRNA and protein is overexpressed in diabetic animals compared to normal. Thus, modulators of HKV expression or activity can be used to treat disorders relating to glucose metabolism, e.g., diabetes. Inhibition of hexokinase V in diabetic or pre-diabetic individuals can, e.g., increase the sensitivity of insulin secretion to glucose. Without intending to limit the invention to a particular mechanism of action, specific inhibition of HKV in diabetic individuals may enhance islet function by restoring the key role of islet GK as a glucose sensor. Modulation of the expression or activity of HKV polypeptides can be beneficial in treating diabetic, pre-diabetic or obese insulin resistant, non-diabetic patients. Furthermore, overexpression HKV polypeptides and/or nucleic acids are indicative of insulin resistance. Thus, detection of HKV can be useful for diagnosis of diabetes and pre-diabetes, e.g., insulin resistance.

General Recombinant Nucleic Acid Methods

In numerous embodiments of the invention, nucleic acids encoding HKV polypeptides will be isolated and cloned using recombinant methods. Such embodiments are used, e.g., to isolate polynucleotides comprising a sequence that is identical or substantially identical to SEQ ID NO:1 for protein expression or for the generation of variants, derivatives, or other HKV sequences. Recombinant methodology is also used to generate expression cassettes, to monitor gene expression, for the isolation or detection of sequences in different species, for diagnostic purposes in a patient, e.g., to detect mutations in an HKV polynucleotide or polypeptide, or to detect expression levels of HKV nucleic acids or polypeptides. In some embodiments, the HKV sequences encoding the polypeptides are operably linked to a heterologous promoter. In one embodiment, the HKV nucleic acids are from any mammal, including, in particular, e.g., a human, a mouse, a rat, etc.

General Recombinant Nucleic Acid Methods

The recombinant methodology used in the invention is routine in the field of recombinant genetics. Basic texts disclosing the general methods include Sambrook & Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et. al., Nucleic Acids Res. 12:6159-6168 (1984). Purification of oligonucleotides is typically by either native acrylamide gel electrophoresis or by anion-exchange HPLC as described in Pearson & Reanier, J. Chrom. 255:137-149 (1983).

The sequencse of the cloned genes and synthetic oligonucleotides can be verified after cloning using, e.g., the chain termination method for sequencing double-stranded templates of Wallace et al., Gene 16:21-26 (1981).

Cloning Methods for the Isolation of Nucleotide Sequences Encoding Desired Proteins

In general, nucleic acids encoding the HKV proteins are cloned from cDNA or genomic libraries. The particular sequences can be identified, e.g., by hybridizing with a probe, the sequence of which can be derived from the sequences disclosed herein, which provide a reference for PCR primers and defines suitable regions for isolating probes specific for HKV polynucleotides. Alternatively, where the sequence is cloned into an expression library, the expressed recombinant protein can be detected immunologically with antisera or purified antibodies made against HKV polypeptides, e.g., SEQ ID NO:2. Methods of constructing cDNA and genomic libraries are well known in the art (see, e.g., Sambrook & Russell, supra; and Ausubel et al., supra).

An alternative method of isolating HKV nucleic acids and their homologs combines the use of synthetic oligonucleotide primers and amplification of an RNA or DNA template (see, e.g., U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify HKV nucleic acid sequences directly from mRNA, from cDNA, from genomic libraries or cDNA libraries. Degenerate oligonucleotides can be designed to amplify HKV homologs using the sequences provided herein. Restriction endonuclease sites can be incorporated into the primers. Polymerase chain reaction or other in vitro amplification methods may also be useful, for example, to clone nucleic acid sequences that code for proteins to be expressed, to make nucleic acids to use as probes for detecting the presence of HKV-encoding mRNA in physiological samples, for nucleic acid sequencing, or for other purposes. Genes amplified by the PCR reaction can be purified from agarose gels and cloned into an appropriate vector.

Synthetic oligonucleotides can be used to construct recombinant HKV genes for use as probes or for expression of protein. This method is performed using a series of overlapping oligonucleotides usually 40-120 bp in length, representing both the sense and nonsense strands of the gene. These DNA fragments are then annealed, ligated and cloned. Alternatively, amplification techniques can be used with precise primers to amplify a specific subsequence of the HKV nucleic acid. The specific subsequence is then ligated into an expression vector.

The nucleic acid encoding a hexokinase V is typically cloned into intermediate vectors before transformation into prokaryotic or eukaryotic cells for replication and/or expression. These intermediate vectors are typically prokaryote vectors, e.g., plasmids, or shuttle vectors.

Optionally, nucleic acids encoding chimeric proteins comprising HKV or domains thereof can be made according to standard techniques. For example, a domain comprising the active site can be covalently linked to a heterologous protein.

To obtain high level expression of an HKV nucleic acid, such as a cDNAs encoding SEQ ID NO:2, one typically subclones a nucleic acid sequence encoding the protein of into an expression vector that contains a promoter, typically a heterologous promoter, to direct transcription, a transcription/translation terminator, and a ribosome binding site for translational initiation. Suitable promoters are well known in the art and described, e.g., in Sambrook & Russell and Ausubel et al. Bacterial expression systems for expressing the protein are available in, e.g., E. coli, Bacillus sp., and Salmonella (Palva et al., Gene 22:229-235 (1983); Mosbach et al., Nature 302:543-545 (1983). Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc. Kits for such expression systems are commercially available.

Eukaryotic expression systems for mammalian cells, yeast, and insect cells are also well known in the art and commercially available. For example, exemplary vectors include SV40-based vectors, papilloma virus vectors, baculovirus vectors, and other vectors allowing expression of proteins under the direction of eukaryotic promoters, e.g., SV40 early promoter, SV40 later promoter, metallothionein promoter, murine mammary tumor virus promoter, Rous sarcoma virus promoter, or other promoters shown effective for expression in eukaryotic cells. In one embodiment, the eukaryotic expression vector is a viral vector, e.g., an adenoviral vector, an adeno-associated vector, or a retroviral vector.

Any of many well known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Russell & Sambrook, supra). It is only necessary that the particular genetic engineering procedure used be capable of successfully introducing at least one gene into the host cell capable of expressing HKV.

After the expression vector is introduced into the cells, the transfected cells are cultured under conditions favoring expression of the protein, which is recovered from the culture using standard techniques identified below.

Transgenic animals, including knockout transgenic animals, that include additional copies of HKV and/or altered or mutated HKV transgenes can also be generated. A “transgenic animal” refers to any animal (e.g. mouse, rat, pig, bird, or an amphibian), preferably a non-human mammal, in which one or more cells contain heterologous nucleic acid introduced using transgenic techniques well known in the art. The nucleic acid is introduced into the cell, directly or indirectly, by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA.

In other embodiments, transgenic animals are produced in which expression of HKV is silenced. Gene knockout by homologous recombination is a method that is commonly used to generate transgenic animals. Transgenic mice can be derived using methodology known to those of skill in the art, see, e.g., Hogan et al., Manipulating the Mouse Embryo: A Laboratory Manual, (1988); Teratocarcinomas and Embryonic Stem Cells: A Practical Approach, Robertson, ed., (1987); and Capecchi et al., Science 244:1288 (1989).

Purification of HKV Proteins

Either naturally occurring or recombinant HKV polypeptides can be purified for use in functional assays. Naturally occurring HKV polypeptides of the invention can be purified from any source (e.g., tissues of an organism expressing an ortholog). Recombinant polypeptides can be purified from any suitable expression system. HKV polypeptides are purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook & Russell, supra).

A number of procedures can be employed when recombinant polypeptides are being purified. For example, proteins having established molecular adhesion properties can be reversibly fused to a polypeptide of the invention. With the appropriate ligand, either protein can be selectively adsorbed to a purification column and then freed from the column in a relatively pure form. The fused protein may be then removed by enzymatic activity. Finally polypeptides can be purified using immunoaffinity columns.

When recombinant proteins are expressed by the transformed bacteria in large amounts, typically after promoter induction, although expression can be constitutive, the proteins may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells typically, but not limited to, by incubation in a buffer of about 100-150 μg/ml lysozyme and 0.1% Nonidet P40, a non-ionic detergent. The cell suspension can be ground using a Polytron grinder (Brinkman Instruments, Westbury, N.Y.). Alternatively, the cells can be sonicated on ice. Alternate methods of lysing bacteria are described in Ausubel et al. and Sambrook et al., both supra, and will be apparent to those of skill in the art.

Alternatively, it is possible to purify proteins from bacteria periplasm. Where the protein is exported into the periplasm of the bacteria, the periplasmic fraction of the bacteria can be isolated by cold osmotic shock in addition to other methods known to those of skill in the art (see, Ausubel et al., supra).

Proteins can also be purified from eukaryotic gene expression systems as described in, e.g., Fernandez and Hoeffler, Gene Expression Systems (1999). In some embodiments, baculovirus expression systems are used to isolate proteins of the invention. Recombinant baculoviruses are generally generated by replacing the polyhedrin coding sequence of a baculovirus with a gene to be expressed (e.g., encoding a polypeptide of the invention). Viruses lacking the polyhedrin gene have a unique plaque morphology making them easy to recognize. In some embodiments, a recombinant baculovirus is generated by first cloning a polynucleotide of interest into a transfer vector (e.g., a pUC based vector) such that the polynucleotide is operably linked to a polyhedrin promoter. The transfer vector is transfected with wildtype DNA into an insect cell (e.g., Sf9, Sf21 or BT1-TN-5B1-4 cells), resulting in homologous recombination and replacement of the polyhedrin gene in the wildtype viral DNA with the polynucleotide of interest. Virus can then be generated and plaque purified. Protein expression results upon viral infection of insect cells. Expressed proteins can be harvested from cell supernatant if secreted, or from cell lysates if intracellular. See, e.g., Ausubel et al. and Fernandez and Hoeffler, supra.

Proteins are purified using standard techniques including, for example, an initial salt fractionation. Other methods that rely on solubility of proteins, such as cold ethanol precipitation, are well known to those of skill in the art and can be used to fractionate complex protein mixtures.

Proteins may also be separated based on a calculated molecular weight using techniques such as ultrafiltration and size separation on a column. The proteins of interest can also be separated from other proteins on the basis of their size, net surface charge, hydrophobicity and affinity for ligands. In addition, antibodies raised against proteins can be conjugated to column matrices and the proteins immunopurified. All of these methods are well known in the art.

Immunoaffinity chromatography using antibodies raised to a variety of affinity tags such as hemagglutinin (HA), FLAG, Xpress, Myc, hexahistidine (His), glutathione S transferase (GST) and the like can be used to purify polypeptides. The His tag will also act as a chelating agent for certain metals (e.g., Ni) and thus the metals can also be used to purify His-containing polypeptides. After purification, the tag is optionally removed by specific proteolytic cleavage.

Detection of Hexokinase V Polynucleotides

Those of skill in the art will recognize that detection of expression of hexokinase V polynucleotides and polypeptides has many uses. For example, as discussed herein, detection of levels of polynucleotides and polypeptides of the invention in a patient can be useful for diagnosing diabetes or a predisposition for at least some of the pathological effects of diabetes. Moreover, detection of gene expression is useful to identify modulators, e.g., inhibitors, of expression of hexokinase V polynucleotides and polypeptides.

Gene expression can be analyzed by techniques known in the art, e.g., reverse transcription and amplification of mRNA, isolation of total RNA or poly A+ RNA, northern blotting, dot blotting, in situ hybridization, RNase protection, probing DNA microchip arrays, and the like, as further described below.

A variety of methods of specific DNA and RNA measurement that use nucleic acid hybridization techniques are known to those of skill in the art (see, Sambrook, supra). Some methods involve an electrophoretic separation (e.g., Southern blot for detecting DNA, and Northern blot for detecting RNA), but measurement of DNA and RNA can also be carried out in the absence of electrophoretic separation (e.g., by dot blot). Southern blot of genomic DNA (e.g., from a human) can be used for screening for restriction fragment length polymorphism (RFLP) to detect the presence of a genetic disorder affecting a polypeptide of the invention.

The selection of a nucleic acid hybridization format is not critical. A variety of nucleic acid hybridization formats are known to those skilled in the art. For example, common formats include sandwich assays and competition or displacement assays. Hybridization techniques are generally described in Hames and Higgins Nucleic Acid Hybridization, A Practical Approach, IRL Press (1985); Gall and Pardue, Proc. Natl. Acad. Sci. U.S.A., 63:378-383 (1969); and John et al. Nature, 223:582-587 (1969).

Detection of a hybridization complex may require the binding of a signal-generating complex to a duplex of target and probe polynucleotides or nucleic acids. Typically, such binding occurs through ligand and anti-ligand interactions as between a ligand-conjugated probe and an anti-ligand conjugated with a signal. The binding of the signal generation complex is also readily amenable to accelerations by exposure to ultrasonic energy.

The label may also allow indirect detection of the hybridization complex. For example, where the label is a hapten or antigen, the sample can be detected by using antibodies. In these systems, a signal is generated by attaching fluorescent or enzyme molecules to the antibodies or in some cases, by attachment to a radioactive label (see, e.g., Tijssen, “Practice and Theory of Enzyme Immunoassays,” Laboratory Techniques in Biochemistry and Molecular Biology, Burdon and van Knippenberg Eds., Elsevier (1985), pp. 9-20).

The probes are typically labeled either directly, as with isotopes, chromophores, lumiphores, chromogens, or indirectly, such as with biotin, to which a streptavidin complex may later bind. Thus, the detectable labels used in the assays of the present invention can be primary labels (where the label comprises an element that is detected directly or that produces a directly detectable element) or secondary labels (where the detected label binds to a primary label, e.g., as is common in immunological labeling). Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P-labeled probes or the like.

Other labels include, e.g., ligands that bind to labeled antibodies, fluorophores, chemiluminescent agents, enzymes, and antibodies that can serve as specific binding pair members for a labeled ligand. An introduction to labels, labeling procedures and detection of labels is found in Polak and Van Noorden Introduction to Immunocytochemistry, 2nd ed., Springer Verlag, NY (1997); and in Haugland Handbook of Fluorescent Probes and Research Chemicals, a combined handbook and catalogue Published by Molecular Probes, Inc. (1996).

In general, a detector that monitors a particular probe or probe combination is used to detect the detection reagent label. Typical detectors include spectrophotometers, phototubes and photodiodes, microscopes, scintillation counters, cameras, film and the like, as well as combinations thereof. Examples of suitable detectors are widely available from a variety of commercial sources known to persons of skill in the art. Commonly, an optical image of a substrate comprising bound labeling moieties is digitized for subsequent computer analysis.

The amount of, for example, an HKV RNA is measured by quantifying the amount of label fixed to the solid support by binding of the detection reagent. Typically, the presence of a modulator during incubation will increase or decrease the amount of label fixed to the solid support relative to a control incubation that does not comprise the modulator, or as compared to a baseline established for a particular reaction type. Means of detecting and quantifying labels are well known to those of skill in the art.

In some embodiments, the target nucleic acid or the probe is immobilized on a solid support. Solid supports suitable for use in the assays of the invention are known to those of skill in the art. As used herein, a solid support is a matrix of material in a substantially fixed arrangement.

A variety of automated solid-phase assay techniques are also appropriate. For instance, very large scale immobilized polymer arrays (VLSIPS™), i.e. Gene Chips or microarrays, available from Affymetrix, Inc. in Santa Clara, Calif. can be used to detect changes in expression levels of a plurality of genes involved in the same regulatory pathways simultaneously. See, Tijssen, supra., Fodor et al. (1991) Science, 251: 767-777; Sheldon et al. (1993) Clinical Chemistry 39(4): 718-719, and Kozal et al. (1996) Nature Medicine 2(7): 753-759. Similarly, spotted cDNA arrays (arrays of cDNA sequences bound to nylon, glass or another solid support) can also be used to monitor expression of a plurality of genes.

Typically, the array elements are organized in an ordered fashion so that each element is present at a specified location on the substrate. Because the array elements are at specified locations on the substrate, the hybridization patterns and intensities (which together create a unique expression profile) can be interpreted in terms of expression levels of particular genes and can be correlated with a particular disease or condition or treatment. See, e.g., Schena et al., Science 270: 467-470 (1995)) and (Lockhart et al., Nature Biotech. 14: 1675-1680 (1996)).

Hybridization specificity can be evaluated by comparing the hybridization of specificity-control polynucleotide sequences to specificity-control polynucleotide probes that are added to a sample in a known amount. The specificity-control target polynucleotides may have one or more sequence mismatches compared with the corresponding polynucleotide sequences. In this manner, whether only complementary target polynucleotides are hybridizing to the polynucleotide sequences or whether mismatched hybrid duplexes are forming is determined.

Hybridization reactions can be performed in absolute or differential hybridization formats. In the absolute hybridization format, polynucleotide probes from one sample are hybridized to the sequences in a microarray format and signals detected after hybridization complex formation correlate to polynucleotide probe levels in a sample. In the differential hybridization format, the differential expression of a set of genes in two biological samples is analyzed. For differential hybridization, polynucleotide probes from both biological samples are prepared and labeled with different labeling moieties. A mixture of the two labeled polynucleotide probes is added to a microarray. The microarray is then examined under conditions in which the emissions from the two different labels are individually detectable. Sequences in the microarray that are hybridized to substantially equal numbers of polynucleotide probes derived from both biological samples give a distinct combined fluorescence (Shalon et al. PCT publication WO95/35505). In some embodiments, the labels are fluorescent labels with distinguishable emission spectra, such as Cy3 and Cy5 fluorophores.

After hybridization, the microarray is washed to remove nonhybridized nucleic acids and complex formation between the hybridizable array elements and the polynucleotide probes is detected. Methods for detecting complex formation are well known to those skilled in the art. In some embodiments, the polynucleotide probes are labeled with a fluorescent label and measurement of levels and patterns of fluorescence indicative of complex formation is accomplished by fluorescence microscopy, such as confocal fluorescence microscopy.

In a differential hybridization experiment, polynucleotide probes from two or more different biological samples are labeled with two or more different fluorescent labels with different emission wavelengths. Fluorescent signals are detected separately with different photomultipliers set to detect specific wavelengths. The relative abundances/expression levels of the polynucleotide probes in two or more samples are obtained.

Typically, microarray fluorescence intensities can be normalized to take into account variations in hybridization intensities when more than one microarray is used under similar test conditions. In some embodiments, individual polynucleotide probe/target complex hybridization intensities are normalized using the intensities derived from internal normalization controls contained on each microarray.

Detection of nucleic acids can also be accomplished, for example, by using a labeled detection moiety that binds specifically to duplex nucleic acids (e.g., an antibody that is specific for RNA-DNA duplexes). One example uses an antibody that recognizes DNA-RNA heteroduplexes in which the antibody is linked to an enzyme (typically by recombinant or covalent chemical bonding). The antibody is detected when the enzyme reacts with its substrate, producing a detectable product. Coutlee et al. (1989) Analytical Biochemistry 181:153-162; Bogulavski (1986) et al. J. Immunol. Methods 89:123-130; Prooijen-Knegt (1982) Exp. Cell Res. 141:397-407; Rudkin (1976) Nature 265:472-473, Stollar (1970) PNAS 65:993-1000; Ballard (1982) Mol. Immunol. 19:793-799; Pisetsky and Caster (1982) Mol. Immunol. 19:645-650; Viscidi et al. (1988) J. Clin. Microbial. 41:199-209; and Kiney et al. (1989) J. Clin. Microbiol. 27:6-12 describe antibodies to RNA duplexes, including homo and heteroduplexes. Kits comprising antibodies specific for DNA:RNA hybrids are available, e.g., from Digene Diagnostics, Inc. (Beltsville, Md.).

In addition to available antibodies, one of skill in the art can easily make antibodies specific for nucleic acid duplexes using existing techniques, or modify those antibodies that are commercially or publicly available. In addition to the art referenced above, general methods for producing polyclonal and monoclonal antibodies are known to those of skill in the art (see, e.g., Paul (ed) Fundamental Immunology, Third Edition Raven Press, Ltd., NY (1993); Coligan Current Protocols in Immunology Wiley/Greene, NY (1991); Harlow and Lane Antibodies: A Laboratory Manual Cold Spring Harbor Press, NY (1989); Stites et al. (eds.) Basic and Clinical Immunology (4th ed.) Lange Medical Publications, Los Altos, Calif., and references cited therein; Goding Monoclonal Antibodies: Principles and Practice (2d ed.) Academic Press, New York, N.Y., (1986); and Kohler and Milstein Nature 256: 495-497 (1975)). Other suitable techniques for antibody preparation include selection of libraries of recombinant antibodies in phage or similar vectors (see, Huse et al. Science 246:1275-1281 (1989); and Ward et al. Nature 341:544-546 (1989)). Specific monoclonal and polyclonal antibodies and antisera will usually bind with a K_{D} of at least about 0.1 μM, preferably at least about 0.01 μM or better, and most typically and preferably, 0.001 μM or better.

The hexokinase V nucleic acids used in this invention can be either positive or negative probes. Positive probes bind to their targets and the presence of duplex formation is evidence of the presence of the target. Negative probes fail to bind to the suspect target and the absence of duplex formation is evidence of the presence of the target. For example, the use of a wild type specific nucleic acid probe or PCR primers may serve as a negative probe in an assay sample where only the nucleotide sequence of interest is present.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBA, Cangene, Mississauga, Ontario) and Q Beta Replicase systems. These systems can be used to directly identify mutants where the PCR or LCR primers are designed to be extended or ligated only when a selected sequence is present. Alternatively, the selected sequences can be generally amplified using, for example, nonspecific PCR primers and the amplified target region later probed for a specific sequence indicative of a mutation. It is understood that various detection probes, including Taqman and molecular beacon probes can be used to monitor amplification reaction products, e.g., in real time.

An alternative means for determining the level of expression of the nucleic acids of the present invention is in situ hybridization. In situ hybridization assays are well known and are generally described in Angerer et al., Methods Enzymol. 152:649-660 (1987). In an in situ hybridization assay, cells, preferentially human cells from the cerebellum or the hippocampus, are fixed to a solid support, typically a glass slide. If DNA is to be probed, the cells are denatured with heat or alkali. The cells are then contacted with a hybridization solution at a moderate temperature to permit annealing of specific probes that are labeled. The probes are preferably labeled with radioisotopes or fluorescent reporters.

Single nucleotide polymorphism (SNP) analysis is also useful for detecting differences between hexokinase V alleles. SNPs linked to genes encoding polypeptides of the invention are useful, for instance, for diagnosis of diabetes or a predisposition to diabetes whose occurrence is linked to the gene sequences of the invention. For example, if an individual carries at least one SNP linked to a disease-associated allele of the gene sequences of the invention, the individual is likely predisposed for one or more of those diseases. If the individual is homozygous for a disease-linked SNP, the individual is particularly predisposed for occurrence of that disease (e.g., diabetes). In some embodiments, the SNP associated with the gene sequences of the invention is located within 300,000; 200,000; 100,000; 75,000; 50,000; or 10,000 base pairs from the gene sequence.

Various real-time PCR methods including, e.g., Taqman or molecular beacon-based assays (e.g., U.S. Pat. Nos. 5,210,015; 5,487,972; Tyagi et al., Nature Biotechnology 14:303 (1996); and PCT WO 95/13399 are useful to monitor for the presence of absence of a SNP. Additional SNP detection methods include, e.g., DNA sequencing, sequencing by hybridization, dot blotting, oligonucleotide array (DNA Chip) hybridization analysis, or are described in, e.g., U.S. Pat. No. 6,177,249; Landegren et al., Genome Research, 8:769-776 (1998); Botstein et al., Am J Human Genetics 32:314-331 (1980); Meyers et al., Methods in Enzymology 155:501-527 (1987); Keen et al., Trends in Genetics 7:5 (1991); Myers et al., Science 230:1242-1246 (1985); and Kwok et al., Genomics 23:138-144 (1994).

Immunodetection of Hexokinase V Polypeptides

In addition to the detection of HKV polynucloetides and gene expression using nucleic acid hybridization technology, one can also use immunoassays to detect HKV polypeptides. Immunoassays can be used to qualitatively or quantitatively analyze polypeptides of the invention. A general overview of the applicable technology can be found, e.g., in Harlow & Lane, Antibodies: A Laboratory Manual (1988) and Harlow & Lane, Using Antibodies (1999).

Antibodies to HKV Proteins or Other Immunogens

Methods for producing polyclonal and monoclonal antibodies that react specifically with an HKV protein or other immunogen are known to those of skill in the art (see, e.g., Coligan, supra; and Harlow and Lane, supra; Stites et al., supra and references cited therein; Goding, supra; and Kohler and Milstein Nature, 256:495-497 (1975)). Such techniques include antibody preparation by selection of antibodies from libraries of recombinant antibodies in phage or similar vectors (see, Huse et al., supra; and Ward et al., supra). For example, in order to produce antisera for use in an immunoassay, the protein of interest or an antigenic fragment thereof, is isolated as described herein. For example, a recombinant HKV protein is produced in a transformed cell line. An inbred strain of mice or rabbits is immunized with the protein using a standard adjuvant, such as Freund's adjuvant, and a standard immunization protocol. Alternatively, a synthetic peptide derived from the HKV sequences disclosed herein is conjugated to a carrier protein and used as an immunogen.

Polyclonal sera are collected and titered against the immunogen in an immunoassay, for example, a solid phase immunoassay with the immunogen immobilized on a solid support. Polyclonal antisera with a titer of 10⁴ or greater are selected and tested for their crossreactivity against proteins other than the polypeptides of the invention or even other homologous proteins from other organisms, using a competitive binding immunoassay. Specific monoclonal and polyclonal antibodies and antisera will usually bind with a K_{D} of at least about 0.1 mM, more usually at least about 1 μM, preferably at least about 0.1 μM or better, and most preferably, 0.01 μM or better.

Recombinant protein is the preferred immunogen for the production of monoclonal or polyclonal antibodies. Naturally occurring protein may also be used either in pure or impure form. Synthetic peptides made using the protein sequences described herein may also be used as an immunogen for the production of antibodies to the protein. Recombinant protein can be expressed in eukaryotic or prokaryotic cells and purified as generally described supra. The product is then injected into an animal capable of producing antibodies. Either monoclonal or polyclonal antibodies may be generated for subsequent use in immunoassays to measure the protein.

Methods of production of polyclonal antibodies are known to those of skill in the art. In brief, an immunogen, preferably a purified protein, is mixed with an adjuvant and animals are immunized. The animal's immune response to the immunogen preparation is monitored by taking test bleeds and determining the titer of reactivity to polypeptides of the invention. When appropriately high titers of antibody to the immunogen are obtained, blood is collected from the animal and antisera are prepared. Further fractionation of the antisera to enrich for antibodies reactive to the protein can be done if desired (see, Harlow and Lane, supra).

Monoclonal antibodies may be obtained using various techniques familiar to those of skill in the art. Typically, spleen cells from an animal immunized with a desired antigen are immortalized, commonly by fusion with a myeloma cell (see, Kohler and Milstein, Eur. J. Immunol. 6:511-519 (1976)). Alternative methods of immortalization include, e.g., transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences that encode a monoclonal antibody or a binding fragment thereof by screening a DNA library from human B cells according to the general protocol outlined by Huse et al., supra.

Once target immunogen-specific antibodies are available, the immunogen can be measured by a variety of immunoassay methods with qualitative and quantitative results available to the clinician. For a review of immunological and immunoassay procedures in general see, Stites, supra. Moreover, the immunoassays of the present invention can be performed in any of several configurations, which are reviewed extensively in Maggio Enzyme Immunoassay, CRC Press, Boca Raton, Fla. (1980); Tijssen, supra; and Harlow and Lane, supra.

Immunoassays to measure target proteins in a human sample may use a polyclonal antiserum that was raised to full-length polypeptides of the invention or a fragment thereof. This antiserum is selected to have low cross-reactivity against other proteins and any such cross-reactivity is removed by immunoabsorption prior to use in the immunoassay.

Immunoassays

In some embodiments, a protein of interest is detected and/or quantified using any of a number of well-known immunological binding assays (see, e.g., U.S. Pat. Nos. 4,366,241; 4,376,110; 4,517,288; and 4,837,168). For a review of the general immunoassays, see also Asai Methods in Cell Biology Volume 37: Antibodies in Cell Biology, Academic Press, Inc. NY (1993); Stites, supra. Immunological binding assays (or immunoassays) typically utilize a “capture agent” to specifically bind to and often immobilize the analyte (e.g., full-length polypeptides of the present invention, or antigenic subsequences thereof). The capture agent is a moiety that specifically binds to the analyte. The antibody may be produced by any of a number of means well known to those of skill in the art and as described above.

Immunoassays also often utilize a labeling agent to bind specifically to and label the binding complex formed by the capture agent and the analyte. The labeling agent may itself be one of the moieties comprising the antibody/analyte complex. Alternatively, the labeling agent may be a third moiety, such as another antibody, that specifically binds to the antibody/protein complex.

In a preferred embodiment, the labeling agent is a second antibody bearing a label. Alternatively, the second antibody may lack a label, but it may, in turn, be bound by a labeled third antibody specific to antibodies of the species from which the second antibody is derived. The second antibody can be modified with a detectable moiety, such as biotin, to which a third labeled molecule can specifically bind, such as enzyme-labeled streptavidin.

Other proteins capable of specifically binding immunoglobulin constant regions, such as protein A or protein G, can also be used as the label agents. These proteins are normal constituents of the cell walls of streptococcal bacteria. They exhibit a strong non-immunogenic reactivity with immunoglobulin constant regions from a variety of species (see, generally, Kronval, et al. J. Immunol., 111:1401-1406 (1973); and Akerstrom, et al. J. Immunol., 135:2589-2542 (1985)).

Throughout the assays, incubation and/or washing steps may be required after each combination of reagents. Incubation steps can vary from about 5 seconds to several hours, preferably from about 5 minutes to about 24 hours. The incubation time will depend upon the assay format, analyte, volume of solution, concentrations, and the like. Usually, the assays will be carried out at ambient temperature, although they can be conducted over a range of temperatures, such as 10° C. to 40° C.

Immunoassays for detecting HKV proteins or other analytes of interest from tissue samples may be either competitive or noncompetitive. Noncompetitive immunoassays are assays in which the amount of captured protein or analyte is directly measured. In one preferred “sandwich” assay, for example, the capture agent (e.g., antibodies specific for the polypeptides of the invention) can be bound directly to a solid substrate where it is immobilized. These immobilized antibodies then capture the polypeptide present in the test sample. The polypeptide of the invention thus immobilized is then bound by a labeling agent, such as a second labelled antibody specific for the polypeptide. Alternatively, the second antibody may lack a label, but it may, in turn, be bound by a labeled third antibody specific to antibodies of the species from which the second antibody is derived. The second can be modified with a detectable moiety, such as biotin, to which a third labeled molecule can specifically bind, such as enzyme-labeled streptavidin.

In some embodiments, western blot (immunoblot) analysis is used to detect and quantify the presence of a polypeptide of the invention in the sample. The technique generally comprises separating sample proteins by gel electrophoresis, transferring the separated proteins to a suitable solid support and incubating the sample with the antibodies that specifically bind the protein of interest. These antibodies may be directly labeled or alternatively may be subsequently detected using labeled antibodies (e.g., labeled sheep anti-mouse antibodies) that specifically bind to the antibodies against the protein of interest.

Other assay formats include liposome immunoassays (LIA), which use liposomes designed to bind specific molecules (e.g., antibodies) and release encapsulated reagents or markers. The released chemicals are then detected according to standard techniques (see, Monroe et al. (1986) Amer. Clin. Prod. Rev. 5:34-41).

In competitive assays, the amount of protein or analyte present in the sample is measured indirectly by measuring the amount of an added (exogenous) protein or analyte displaced (or competed away) from a specific capture agent (e.g., antibodies specific for a polypeptide of the invention) by the protein or analyte present in the sample. The amount of immunogen bound to the antibody is inversely proportional to the concentration of immunogen present in the sample. In a particularly preferred embodiment, the antibody is immobilized on a solid substrate. The amount of analyte may be detected by providing a labeled analyte molecule. It is understood that labels can include, e.g., radioactive labels as well as peptide or other tags that can be recognized by detection reagents such as antibodies.

Immunoassays in the competitive binding format can be used for cross-reactivity determinations. For example, the protein encoded by the sequences described herein can be immobilized on a solid support. Proteins are added to the assay and compete with the binding of the antisera to the immobilized antigen. The ability of the above proteins to compete with the binding of the antisera to the immobilized protein is compared to that of the protein encoded by any of the sequences described herein. The percent cross-reactivity for the above proteins is calculated, using standard calculations. Those antisera with less than 10% cross-reactivity with each of the proteins listed above are selected and pooled. The cross-reacting antibodies are optionally removed from the pooled antisera by immunoabsorption with the considered proteins, e.g., distantly related homologs.

The immunoabsorbed and pooled antisera are then used in a competitive binding immunoassay as described above to compare a second protein, thought to be perhaps a protein of the present invention, to the immunogen protein. In order to make this comparison, the two proteins are each assayed at a wide range of concentrations and the amount of each protein required to inhibit 50% of the binding of the antisera to the immobilized protein is determined. If the amount of the second protein required is less than 10 times the amount of the protein partially encoded by a sequence herein that is required, then the second protein is said to specifically bind to an antibody generated to an immunogen consisting of the target protein.

Labels

The particular label or detectable group used in various assays is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most labels useful in such methods can be applied to the present invention. Thus, a label is any composition detectable by spectroscopic, photochemical, biochemical, immunochemical, electrical, optical or chemical means. Useful labels in the present invention include magnetic beads (e.g., Dynabeads™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

The label may be coupled directly or indirectly to the desired component of the assay according to methods well known in the art. As indicated above, a wide variety of labels may be used, with the choice of label depending on the sensitivity required, the ease of conjugation with the compound, stability requirements, available instrumentation, and disposal provisions.

Non-radioactive labels are often attached by indirect means. The molecules can also be conjugated directly to signal generating compounds, e.g., by conjugation with an enzyme or fluorescent compound. A variety of enzymes and fluorescent compounds can be used with the methods of the present invention and are well-known to those of skill in the art (for a review of various labeling or signal producing systems which may be used, see, e.g., U.S. Pat. No. 4,391,904).

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected directly by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need to be labeled and the presence of the target antibody is detected by simple visual inspection.

Identification of Modulators of Hexokinase V

Inhibitors of HK V, i.e., inhibitors of HK V activity, or expression, are useful for treating a number of human diseases relating to glucose metabolism, including diabetes. For example, administration of inhibitors can be used to treat diabetic patients or prediabetic individuals to prevent progression, and therefore symptoms, associated with diabetes (including insulin resistance).

A. Agents that Modulate Hexokinase V Polypeptides

The agents tested as modulators of polypeptides of the invention can be any small chemical compound, or a biological entity, such as a protein, sugar, nucleic acid or lipid. Typically, test compounds will be small chemical molecules and peptides. Essentially any chemical compound can be used as a potential modulator or ligand in the assays of the invention, although most often compounds that can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). Modulators also include agents designed to reduce the level of mRNA encoding an HKV polypeptide (e.g., antisense molecules, ribozymes, DNAzymes, small inhibitory RNAs and the like) or the level of translation from an mRNA (e.g., translation blockers such as an antisense molecules that are complementary to translation start or other sequences on an mRNA molecule). Modulators can also be variants or mutant proteins of an HKV polypeptide. It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

In some embodiments, high throughput screening methods involve providing a combinatorial chemical or peptide library containing a large number of potential therapeutic compounds (potential modulator compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

B. Methods of Screening for Modulators of the Polypeptides of the Invention

A number of different screening protocols can be utilized to identify agents that modulate the level of expression or activity of a polynucleotide of a polypeptide of the invention in cells, particularly mammalian cells, and especially human cells. In general terms, the screening methods involve screening a plurality of agents to identify an agent that modulates the activity of a polypeptide of the invention by, e.g., binding to the polypeptide, preventing an inhibitor or activator from binding to the polypeptide, increasing association of an inhibitor or activator with the polypeptide, or activating or inhibiting expression of the polypeptide.

Any cell expressing a full-length polypeptide of the invention or a fragment thereof can be used to identify modulators. In some embodiments, the cells are eukaryotic cells lines (e.g., CHO or HEK293) transformed to express a heterologous hexokinase V polypeptide. In some embodiments, a cell expressing an endogenous HKV polypeptide, e.g., a kidney cell or a pancreatic cell, is used in screens. In other embodiments, modulators are screened for their ability to effect insulin responses, e.g., gluocose-stimulated insulin release.

1. Polypeptide Binding Assays

Preliminary screens can be conducted by screening for agents capable of binding to HK V polypeptides, as at least some of the agents so identified are likely modulators of a polypeptide of the invention. Binding assays are also useful, e.g., for identifying endogenous proteins that interact with HK V. For example, antibodies or other molecules that bind polypeptides of the invention can be identified in binding assays.

Binding assays usually involve contacting an HKV polypeptide with one or more test agents and allowing sufficient time for the protein and test agents to form a binding complex. Any binding complexes formed can be detected using any of a number of established analytical techniques. Protein binding assays include, but are not limited to, methods that measure co-precipitation or co-migration on non-denaturing SDS-polyacrylamide gels, and co-migration on Western blots (see, e.g., Bennet, J. P. and Yamamura, H. I. (1985) “Neurotransmitter, Hormone or Drug Receptor Binding Methods,” in Neurotransmitter Receptor Binding (Yamamura, H. I., et al., eds.), pp. 61-89. Other binding assays involve the use of mass spectrometry or NMR techniques to identify molecules bound the HKV polypeptide or displacement of labeled substrates. The HKV polypeptides used in these assays can be naturally expressed, cloned or synthesized.

In addition, mammalian or yeast two-hybrid approaches (see, e.g., Bartel, P. L. et. al. Methods Enzymol, 254:241 (1995)) can be used to identify polypeptides or other molecules that interact or bind to HKV when expressed together in a host cell.

2. Polypeptide Activity

Hexokinase V activity can be assessed using a variety of in vitro and in vivo assays to determine functional, chemical, and physical effects. These assays include monitoring, for example, catalytic phosphorylation of a sugar, e.g., D-glucose, D-fructose, 5-keto-D-fructose, 2-deoxy-D-glucose, D-mannose and D-glucosamine; or transfer of a phosphate group from a phosphoryl donor such as dATP, ITP, or MgATP. An exemplary hexokinase activity can be performed by a colorimetric method in which the activity of HKV is coupled with the subsequent reduction of NADP to NADPH, which can be detected by increases in absorbance or fluorescence (see, e.g., Palma et al., Protein Expr. Purif 22:38-44, 2001; Tsai & Chen, Biochem. Cell Biol. 76:107-13, 1998). Such an assay is described in more detail in the “Examples” section.

Alternatively, assays formatted for highthroughput use can be used. For example, kinases catalyze the transfer of a gamma-phosphoryl group from ATP to an appropriate hydroxyl acceptor with the release of a proton. An assay based on the detection of this proton using an appropriately matched buffer/indicator system may therefore be used to detect activity (see, e.g., Chapman & Wong Bioorg Med Chem 10:551-5, 2002).

The hexokinase V polypeptide of the assay will be selected from a polypeptide with substantial identity to a sequence of SEQ ID NO:2 or other conservatively modified variants thereof. Generally, the amino acid sequence identity will be at least 70%, optionally at least 85%, optionally at least 90-95% to the HKV polypeptides exemplified herein, or the polypeptide will have at least 10 contiguous amino acids, more often 20, 25, 30, 25, 50, or 100 contiguous amino acids of SEQ ID NO:2. Optionally, the HKV polypeptide used in activity assays will comprise a fragment of a polypeptide of the invention, such as a kinase domain and the like. Either a polypeptide of the invention or a domain thereof can be covalently linked to a heterologous protein to create a chimeric protein used in the assays described herein.

Modulators of hexokinase V activity are tested using either recombinant or naturally occurring polypeptides. The protein can be isolated, expressed in a cell, expressed in a membrane derived from a cell, expressed in tissue or in an animal, either recombinant or naturally occurring. For example, tissue slices, dissociated cells, e.g., from tissues expressing polypeptides of the invention, transformed cells, or membranes can be used. Modulation is tested using one of the in vitro or in vivo assays described herein.

Modulator binding to polypeptides of the invention, a domain, or chimeric protein can be tested in solution, in a bilayer membrane, attached to a solid phase, in a lipid monolayer, or in vesicles. Binding of a modulator can be tested using, e.g., changes in spectroscopic characteristics (e.g., fluorescence, absorbance, refractive index), hydrodynamic (e.g., shape), chromatographic, or solubility properties.

Samples or assays that are treated with a potential modulator (e.g., a “test compound”) are compared to control samples without the test compound, to examine the extent of modulation. Control samples (untreated with candidate compounds are assigned a relative activity value of 100. Inhibition of the polypeptides of the invention is achieved when the activity value relative to the control is about 90%, optionally 50%, optionally 25-0%. Activation of the polypeptides of the invention is achieved when the activity value relative to the control is 110%, optionally 150%, 200%, 300%, 400%, 500%, or 1000-2000%.

3. Expression Assays

Screening assays for a compound that modulates the expression of HKV polynucleotides and polypeptides are also provided. Screening methods generally involve conducting cell-based assays in which test compounds are contacted with one or more cells expressing HKV, and then detecting an increase or decrease in expression (either transcript or translation product). Assays can be performed with any cells that express a hexokinase V polypeptide. Some assays may employ cells that overexpress hexokinase V in comparison to normal cells, e.g., a diabetic pancreatic cell.

Expression can be detected in a number of different ways. As described infra, the expression level of an HKV polynucleotide can be determined by probing the mRNA expressed in a cell with a probe that specifically hybridizes with an HKV transcript (or complementary nucleic acid derived therefrom). Alternatively, an HKV polypeptide can be detected using immunological methods, e.g., an assay in which a cell lysate is probed with antibodies that specifically bind to the polypeptide.

The level of expression or activity of the HKV polynucleotide or polypeptide can be compared to a baseline value. The baseline value can be a value for a control sample or a statistical value that is representative of expression levels of the HKV polynucleotide or polypeptide for a control population (e.g., non-diabetic individuals as described herein) or cells (e.g., tissue culture cells not exposed to a modulator). Negative controls can include cells that do not express HKV. Such cells generally are otherwise substantially genetically the same as the test cells.

Reporter systems can also be used to identify modulators of HKV expression. A variety of different types of cells can be utilized in reporter assays. Cells that do not endogenously express an HKV polypeptide can be prokaryotic, but are preferably eukaryotic. The eukaryotic cells can be any of the cells typically utilized in generating cells that harbor recombinant nucleic acid constructs. Exemplary eukaryotic cells include, but are not limited to, yeast, and various higher eukaryotic cells such as the HEK293, HepG2, COS, CHO and HeLa cell lines.

Various controls can be conducted to ensure that an observed activity is authentic including running parallel reactions with cells that lack the reporter construct or by not contacting a cell harboring the reporter construct with test compound. Compounds can also be further validated as described below.

4. Validation

Agents that are initially identified by any of the foregoing screening methods can be further tested to validate the activity. Modulators that are selected for further study can be tested on a variety of cells, e.g., pancreatic cells such as the beta cell lines HIT-T15, RiNm5, betaTC3, betaHC9, and INS1. Cells that have been engineered to express hexokinase V may also be used. For example, fibroblasts that overexpress HKV may be used to further validate the activity of the candidate modulator. In an example of such an analysis, cells that express HKV are pre-incubated with the modulators and tested for acute (e.g., up to 4 hours) and chronic (overnight) effects of the inhibitor on glucose-stimulated insulin secretion.

Other assays that can be used to confirm the effects of the modulator include indirects assays, such as those that test for the candidate modulator for the ability to inhibit glucose utilization. Such an assay can also determine the effects of HKV inhibition on overall glucose metabolism.

Following such studies, validity of the modulators is tested in suitable animal models. The basic format of such methods involves administering a lead compound identified during an initial screen to an animal that serves as a model for humans and then determining if expression or activity of hexokinase V is in fact modulated.

The effect of the compound will be assessed in either diabetic animals or in diet-induced insulin resistant animals. The blood glucose and insulin levels will be determined. The animal models utilized in validation studies generally are mammals of any kind. Specific examples of suitable animals include, but are not limited to, primates, mice and rats. For example, monogenic models of diabetes (e.g., ob/ob and db/db mice, Zucker rats and Zucker Diabetic Fatty rats etc) or polygenic models of diabetes (e.g., OLETF rats, GK rats, NSY mice, and KK mice) can be useful for validating modulation of a polypeptide of the invention in a diabetic or insulin resistant animal. In addition, transgenic animals expressing human hexokinase V polypeptides can be used to further validate drug candidates.

Compounds are typically selected that increase the sensitivity of the animals to glucose levels in terms of secreting insulin. Sensitivity can be assessed using a number of measures. Generally, such a test involves determining levels of insulin secreted in response to particular levels of blood glucose. Other assays to assess insulin sensitivity and islet function include fasting blood glucose assays, fasting insulin level assays, assessment of glucose levels during an oral or intraperitoneal glucose tolerance test, assessment of insulin or C-peptide levels during an oral or intraperitoneal glucose tolerance test. Other secretagogues, e.g., arginine or glyburide can also be used to test for the glucose specificity of the improvement in islet function.

C. Solid Phase and Soluble High Throughput Assays

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100 to about 1500 different compounds. It is possible to assay several different plates per day; assay screens for up to about 6,000-20,000 or more different compounds are possible using the integrated systems of the invention. In addition, microfluidic approaches to reagent manipulation can be used.

A molecule of interest (e.g., a hexokinase polypeptide or polynucleotide, or a modulator thereof) can be bound to the solid-state component, directly or indirectly, via covalent or non-covalent linkage, e.g., via a tag. The tag can be any of a variety of components. In general, a molecule that binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, poly-His, etc.) Antibodies to molecules with natural binders such as biotin are also widely available and appropriate tag binders (see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody that recognizes the first antibody. In addition to antibody-antigen interactions, receptor-ligand interactions are also appropriate as tag and tag-binder pairs, such as agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherin family, the integrin family, the selectin family, and the like; see, e.g., Pigott & Power, The Adhesion Molecule Facts Book I (1993)). Similarly, toxins and venoms, viral epitopes, hormones (e.g., opiates, steroids, etc.), intracellular receptors (e.g., which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly-gly sequences of between about 5 and 200 amino acids. Such flexible linkers are known to those of skill in the art. For example, poly(ethelyne glycol) linkers are available from Shearwater Polymers, Inc., Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent that fixes a chemical group to the surface that is reactive with a portion of the tag binder. For example, groups that are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature (see, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank and Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

The invention provides in vitro assays for identifying, in a high throughput format, compounds that can modulate the expression or activity of hexokinase V. Control reactions that measure HKV activity in a cell in a reaction that does not include a potential modulator are optional, as the assays are highly uniform. Such optional control reactions are appropriate and increase the reliability of the assay. Accordingly, in some embodiments, the methods of the invention include such a control reaction. For each of the assay formats described, “no modulator” control reactions that do not include a modulator provide a background level of binding activity.

In some assays it will be desirable to have positive controls. At least two types of positive controls are appropriate. First, a known activator of hexokinase V can be incubated with one sample of the assay, and the resulting increase in signal resulting from an increased expression level or activity of a hexokinase V polypeptide or polynucleotide are determined according to the methods herein. Second, a known inhibitor of a polypeptide or a polynucleotide of the invention can be added, and the resulting decrease in signal for the expression or activity of the hexokinase V polypeptide or polynucleotide can be similarly detected. It will be appreciated that modulators can also be combined with activators or inhibitors to find modulators that inhibit the increase or decrease that is otherwise caused by the presence of the known modulator of an HKV polypeptide or polynucleotide.

Compositions, Kits and Integrated Systems

The invention provides compositions, kits and integrated systems for practicing the assays described herein using hexokinase V nucleic acids or polypeptides, antibodies, etc.

The invention provides assay compositions for use in solid phase assays; such compositions can include, for example, one or more nucleic acids encoding HKV immobilized on a solid support, and a labeling reagent. In each case, the assay compositions can also include additional reagents that are desirable for hybridization. Modulators of HKV expression or activity can also be included in the assay compositions.

The invention also provides kits for carrying out the assays described herein. The kits typically include a probe that comprises an antibody that specifically binds an HKV polypeptide or a polynucleotide sequence encoding an HKV polypeptide, and a label for detecting the presence of the probe. Kits can include any of the compositions noted above, and optionally further include additional components such as instructions to practice a high-throughput method of assaying for an effect on HKV expression or activity, one or more containers or compartments (e.g., to hold the probe, labels, or the like), a control modulator of HKV expression or activity, a robotic armature for mixing kit components or the like.

The invention also provides integrated systems for high-throughput screening of potential modulators for an effect on hexokinase V expression or activity. The systems can include a robotic armature which transfers fluid from a source to a destination, a controller which controls the robotic armature, a label detector, a data storage unit which records label detection, and an assay component such as a microtiter dish comprising a well having a reaction mixture or a substrate comprising a fixed nucleic acid or immobilization moiety.

A number of robotic fluid transfer systems are available, or can easily be made from existing components. For example, a Zymate XP (Zymark Corporation; Hopkinton, Mass.) automated robot using a Microlab 2200 (Hamilton; Reno, Nev.) pipetting station can be used to transfer parallel samples to 96 well microtiter plates to set up several parallel simultaneous binding assays.

Optical images viewed (and, optionally, recorded) by a camera or other recording device (e.g., a photodiode and data storage device) are optionally further processed in any of the embodiments herein, e.g., by digitizing the image and storing and analyzing the image on a computer. A variety of commercially available peripheral equipment and software is available for digitizing, storing and analyzing a digitized video or digitized optical image.

One conventional system carries light from the specimen field to a cooled charge-coupled device (CCD) camera, in common use in the art. A CCD camera includes an array of picture elements (pixels). The light from the specimen is imaged on the CCD. Particular pixels corresponding to regions of the specimen (e.g., individual hybridization sites on an array of biological polymers) are sampled to obtain light intensity readings for each position. Multiple pixels are processed in parallel to increase speed. The apparatus and methods of the invention are easily used for viewing any sample, e.g., by fluorescent or dark field microscopic techniques.

Administration and Pharmaceutical Compositions

Hexokinase V modulators, e.g., inhibitors can be administered directly to the mammalian subject for modulation of activity of a polypeptide of the invention in vivo. Administration is by any of the routes normally used for introducing a modulator compound into ultimate contact with the tissue to be treated and is well known to those of skill in the art. Although more than one route can be used to administer a particular composition, a particular route can often provide a more immediate and more effective reaction than another route.

The pharmaceutical compositions of the invention may comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 17^{th} ed. 1985)).

Inhibitors of the expression or activity of HK V alone or in combination with other suitable components, can be prepared for injection or for use in a pump device. Pump devices (also known as “insulin pumps”) are commonly used to administer insulin to patients and therefore can be easily adapted to include compositions of the present invention. Manufacturers of insulin pumps include Animas, Disetronic and MiniMed.

HK V inhibitors, alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, orally, nasally, topically, intravenously, intraperitoneally, or intrathecally. The formulations of compounds can be presented in unit-dose or multi-dose sealed containers, such as ampoules and vials. Solutions and suspensions can be prepared from sterile powders, granules, and tablets of the kind previously described. The modulators can also be administered as part of a prepared food or drug.

The dose administered to a patient, in the context of the present invention should be sufficient to induce a beneficial response in the subject over time. The optimal dose level for any patient will depend on a variety of factors including the efficacy of the specific modulator employed, the age, body weight, physical activity, and diet of the patient, on a possible combination with other drugs, and on the severity of the case of diabetes. It is recommended that the daily dosage of the modulator be determined for each individual patient by those skilled in the art in a similar way as for known insulin compositions. The size of the dose also will be determined by the existence, nature, and extent of any adverse side-effects that accompany the administration of a particular compound or vector in a particular subject.

In determining the effective amount of the modulator to be administered a physician may evaluate circulating plasma levels of the modulator, modulator toxicity, and the production of anti-modulator antibodies. In general, the dose equivalent of a modulator is from about 1 ng/kg to 10 mg/kg for a typical subject.

For administration, modulators of the present invention can be administered at a rate determined by the LD-50 of the modulator, and the side-effects of the modulator at various concentrations, as applied to the mass and overall health of the subject. Administration can be accomplished via single or divided doses.

The compounds of the present invention can also be used effectively in combination with one or more additional active agents depending on the desired target therapy (see, e.g., Turner, N. et al. Prog. Drug Res. (1998) 51: 33-94; Haffner, S. Diabetes Care (1998) 21: 160-178; and DeFronzo, R. et al. (eds.), Diabetes Reviews (1997) Vol. 5 No. 4). A number of studies have investigated the benefits of combination therapies with oral agents (see, e.g., Mahler, R., J. Clin. Endocrinol. Metab. (1999) 84: 1165-71; United Kingdom Prospective Diabetes Study Group: UKPDS 28, Diabetes Care (1998) 21: 87-92; Bardin, C. W. (ed.), Current Therapy In Endocrinology And Metabolism, 6th Edition (Mosby—Year Book, Inc., St. Louis, Mo. 1997); Chiasson, J. et al., Ann. Intern. Med. (1994) 121: 928-935; Coniff, R. et al., Clin. Ther. (1997) 19: 16-26; Coniff, R. et al., Am. J. Med. (1995) 98: 443-451; and Iwamoto, Y. et al., Diabet. Med. (1996) 13 365-370; Kwiterovich, P. Am. J. Cardiol (1998) 82(12A): 3U-17U). These studies indicate that modulation of diabetes, among other diseases, can be further improved by the addition of a second agent to the therapeutic regimen. Combination therapy includes administration of a single pharmaceutical dosage formulation that contains a modulator of the invention and one or more additional active agents, as well as administration of a modulator and each active agent in its own separate pharmaceutical dosage formulation. For example, a modulator and a thiazolidinedione can be administered to the human subject together in a single oral dosage composition, such as a tablet or capsule, or each agent can be administered in separate oral dosage formulations. Where separate dosage formulations are used, a modulator and one or more additional active agents can be administered at essentially the same time (i.e., concurrently), or at separately staggered times (i.e., sequentially). Combination therapy is understood to include all these regimens.

One example of combination therapy can be seen in treating pre-diabetic individuals (e.g., to prevent progression into type 2 diabetes) or diabetic individuals (or treating diabetes and its related symptoms, complications, and disorders), wherein the modulators can be effectively used in combination with, for example, sulfonylureas (such as chlorpropamide, tolbutamide, acetohexamide, tolazamide, glyburide, gliclazide, glynase, glimepiride, and glipizide); biguanides (such as metformin); a PPAR beta delta agonist; a ligand or agonist of PPAR gamma such as thiazolidinediones (such as ciglitazone, pioglitazone (see, e.g., U.S. Pat. No. 6,218,409), troglitazone, and rosiglitazone (see, e.g., U.S. Pat. No. 5,859,037)); PPAR alpha agonists such as clofibrate, gemfibrozil, fenofibrate, ciprofibrate, and bezafibrate; dehydroepiandrosterone (also referred to as DHEA or its conjugated sulphate ester, DHEA-SO4); antiglucocorticoids; TNFα inhibitors; α-glucosidase inhibitors (such as acarbose, miglitol, and voglibose); amylin and amylin derivatives (such as pramlintide, (see, also, U.S. Pat. Nos. 5,902,726; 5,124,314; 5,175,145 and 6,143,718.)); insulin secretogogues (such as repaglinide, gliquidone, and nateglinide (see, also, U.S. Pat. Nos. 6,251,856; 6,251,865; 6,221,633; 6,174,856)), and insulin.

Gene Therapy

Conventional viral and non-viral based gene transfer methods can be used to introduce nucleic acids encoding engineered HKV polypeptides in mammalian cells or target tissues, or alternatively, HKV nucleic acids e.g., inhibitors of HKV activity, e.g., siRNAs or anti-sense RNAs. Such methods can be used to administer HKV nucleic acids in vitro. In some embodiments, the nucleic acids encoding polypeptides of the invention are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11: 167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Böhm (eds) (1995); and Yu et al., Gene Therapy 1: 13-26 (1994).

In some embodiments, small interfering RNAs are administered. In mammalian cells, introduction of long dsRNA (>30 nt) often initiates a potent antiviral response, exemplified by nonspecific inhibition of protein synthesis and RNA degradation. The phenomenon of RNA interference is described and discussed, e.g., in Bass, Nature 411:428-29 (2001); Elbahir et al., Nature 411:494-98 (2001); and Fire et al., Nature 391:806-11 (1998), where methods of making interfering RNA also are discussed. The siRNAs based upon the HKV sequence disclosed herein are less than 100 base pairs, typically 30 bps or shorter, and are made by approaches known in the art. Exemplary siRNAs according to the invention could have up to 29 bps, 25 bps, 22 bps, 21 bps, 20 bps, 15 bps, 10 bps, 5 bps or any integer thereabout or therebetween.

Non-Viral Delivery Methods

Methods of non-viral delivery of nucleic acids encoding engineered polypeptides of the invention include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. No. 5,049,386, U.S. Pat. No. 4,946,787; and U.S. Pat. No. 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Viral Delivery Methods

The use of RNA or DNA viral based systems for the delivery of nucleic acids encoding engineered HKV polypeptides or nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of polypeptides of the invention could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); PCT/US94/05700).

In applications where transient expression of an HKV nucleic acid is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994)). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

pLASN and MFG-S are examples are retroviral vectors that have been used in clinical trials (Dunbar et al., Blood 85:3048-305 (1995); Kohn et al., Nat. Med. 1:1017-102 (1995); Malech et al., PNAS 94:22 12133-12138 (1997)). PA317/pLASN was the first therapeutic vector used in a gene therapy trial. (Blaese et al., Science 270:475-480 (1995)). Transduction efficiencies of 50% or greater have been observed for MFG-S packaged vectors. (Ellem et al., Immunol Immunother. 44(1):10-20 (1997); Dranoff et al., Hum. Gene Ther. 1:111-2(1997).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)).

Replication-deficient recombinant adenoviral vectors (Ad) can be engineered such that a desired nucleic acid replaces the Ad E1a, E1b, and E3 genes; subsequently the replication defector vector is propagated in human 293 cells that supply deleted gene function in trans. Ad vectors can transduce multiply types of tissues in vivo, including nondividing, differentiated cells such as those found in the liver, kidney, muscle, and pancreatic system tissues. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:1 5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by producer cell line that packages a nucleic acid vector into a viral particle. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type, e.g., pancreatic tissue. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., PNAS 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient.

Ex vivo cell transfection for diagnostics, research, or for gene therapy (e.g., via re-infusion of the transfected cells into the host organism) is well known to those of skill in the art. In some embodiments, cells are isolated from the subject organism, transfected with an HKV nucleic acid and re-infused back into the subject organism (e.g., patient). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

Vectors (e.g., retroviruses, adenoviruses, liposomes, etc.) containing therapeutic nucleic acids can be also administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention, as described below (see, e.g., Remington's Pharmaceutical Sciences, 17th ed., 1989).

Diagnosis of Diabetes

The present invention also provides methods of diagnosing diabetes or a predisposition of at least some of the pathologies of diabetes. Diagnosis can involve determination of a genotype of an individual (e.g., with SNPs) and comparison of the genotype with alleles known to have an association with the occurrence of diabetes. Alternatively, diagnosis also involves determining the level of an HKV polypeptide or polynucleotide in a patient and then comparing the level to a baseline or range. Typically, the baseline value is representative of a polypeptide or polynucleotide of the invention in a healthy (e.g., non-diabetic) person.

As discussed above, variation of levels (e.g., low or high levels) of a polypeptide or polynucleotide of the invention compared to the baseline range indicates that the patient is either diabetic or at risk of developing at least some of the pathologies of diabetes (e.g., pre-diabetic). The level of a polypeptide in a Inon-diabetic individual can be a reading from a single individual, but is typically a statistically relevant average from a group of non-diabetic individuals. The level of a polypeptide in a lean individual can be represented by a value, for example in a computer program.

In some embodiments, the level of HKV polypeptide or polynucleotide is measured by taking a blood, urine or tissue sample from a patient and measuring the amount of a polypeptide or polynucleotide of the invention in the sample using any number of detection methods, such as those discussed herein. For instance, fasting and fed blood or urine levels can be tested.

In some embodiments, the baseline level and the level in a non-diabetic sample from an individual, or at least two samples from the same individual, differ by at least about 5%, 10%, 20%, 50%, 75%, 100%, 150%, 200%, 300%, 400%, 500%, 1000% or more. In some embodiments, the sample from the individual is greater by at least one of the above-listed percentages relative to the baseline level. In some embodiments, the sample from the individual is lower by at least one of the above-listed percentages relative to the baseline level.

In some embodiments, the level of an HKV polypeptide or polynucleotide is used to monitor the effectiveness of treatments for diabetes such as thiazolidinediones, metformin, sulfonylureas and other standard therapies. In some embodiments the activity or expression of an HKV polypeptide or polynucleotide is measured prior to and after treatment of diabetic or pre-diabetic patients with antidiabetic therapies as a surrogate marker of clinical effectiveness. For example, the greater the reduction in expression or activity HKV indicates greater effectiveness.

Glucose/insulin tolerance tests can also be used to detect the effect of glucose levels on levels of HKV polypeptides or polynucleotides. In glucose tolerance tests, the patient's ability to tolerate a standard oral glucose load is evaluated by assessing serum and urine specimens for glucose levels. Blood samples are taken before the glucose is ingested, glucose is given by mouth, and blood or urine glucose levels are tested at set intervals after glucose ingestion. Similarly, meal tolerance tests can also be used to detect the effect of insulin or food, respectively, on levels of HKV.

### EXAMPLES

### Example 1

### Expression of Hexokinase V in the Pancreas

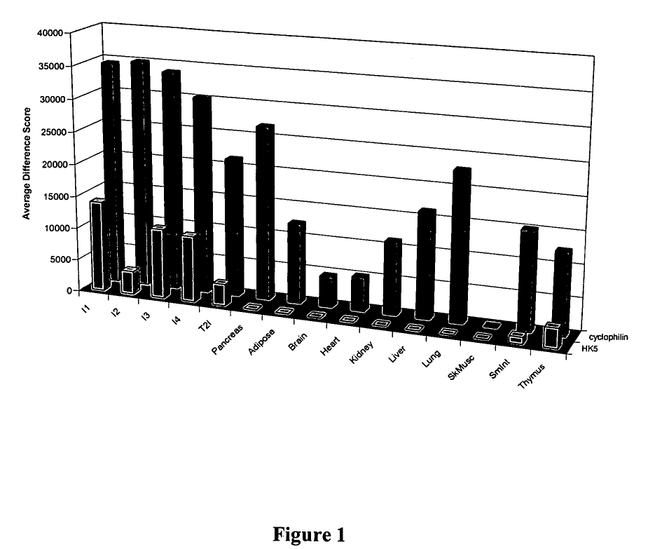

Custom human islet endocrine oligonucleotide arrays were screened with human pancreatic islet mRNA samples and samples from other human tissues to identify genes for which expression is specific to or highly enriched in islets. One of these genes was represented by a set of oligonucleotides that displayed the enriched-in-islet pattern shown in FIG. 1. These oligonucleotides were derived from a cluster of transcript sequences from the library sequencing effort that preceded the design of the genechips that contained a coding sequence similar, but not identical, to human hexokinase I and II. A complete coding sequence for hexokinase V was assembled and used to design PCR primer. These primers amplified hexokinase V cDNA from human kidney mRNA. Like hexokinases I, II and III, hexokinase V is composed of two similar domains in tandem.

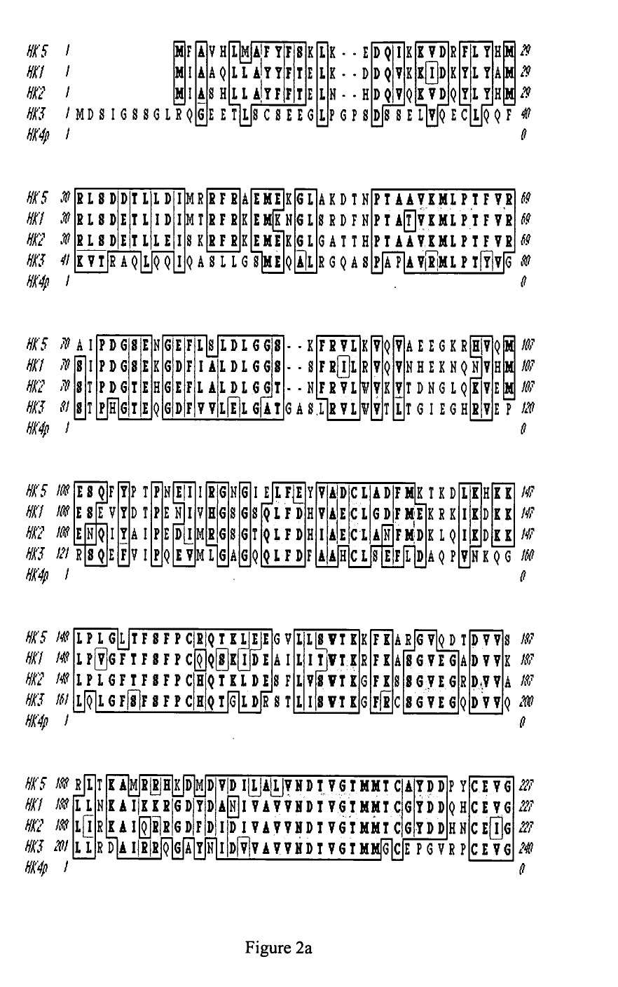

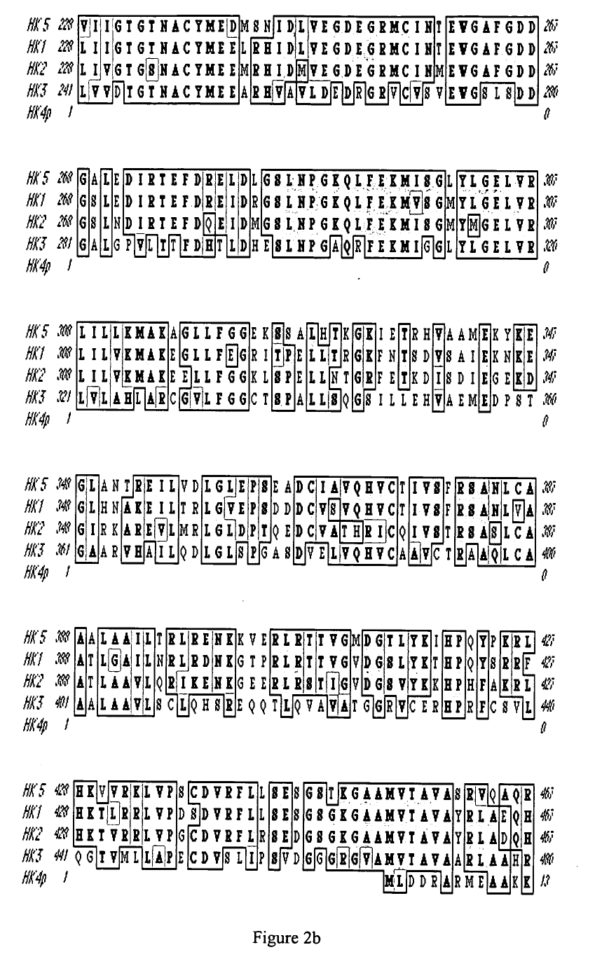

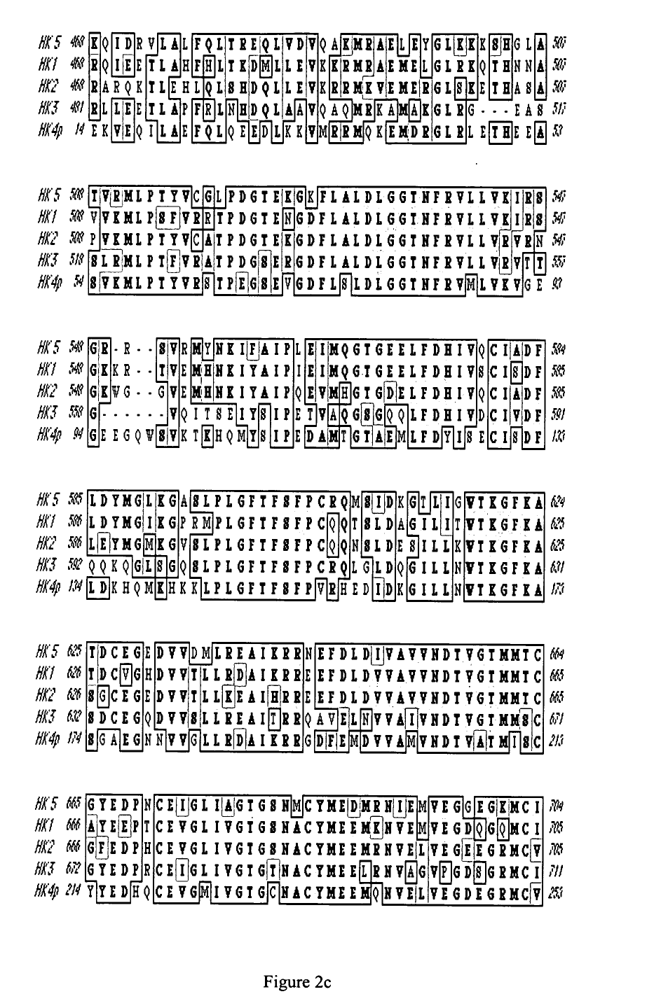

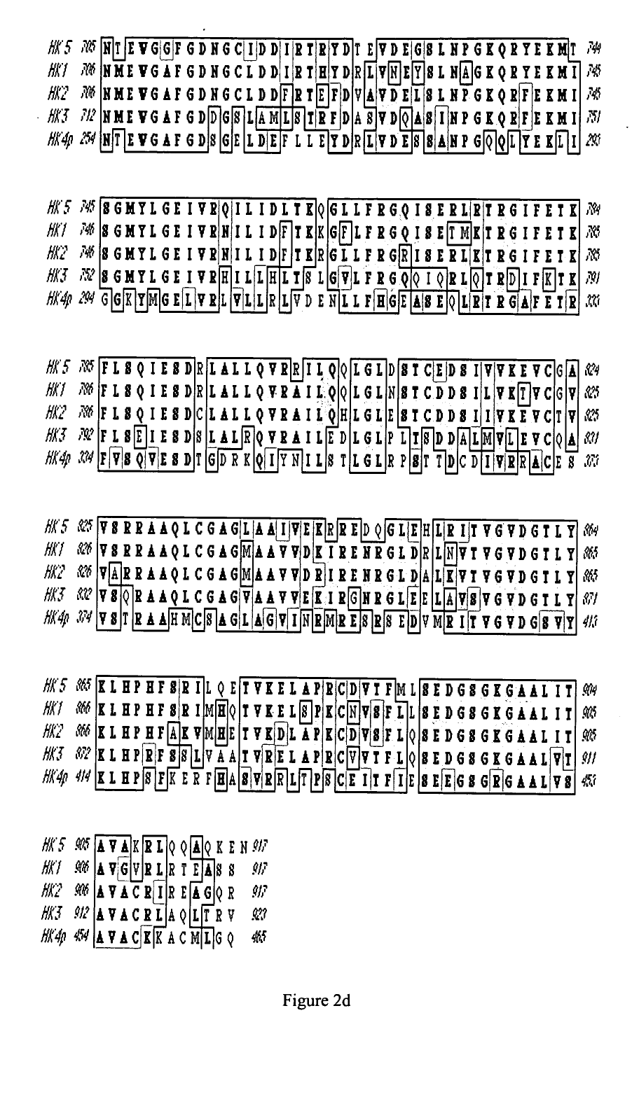

The amino acid sequence of human hexokinase V is 71% identical to hexokinase I, 68% identical to hexokinase II, 54% identical to hexokinase III and 53% identical to the pancreatic islet form of glucokinase (hexokinase IV) (FIGS. 2a-2d). Hexokinase V and hexokinase I share a bipartite domain structure and 71% amino acid identity. In hexokinase I, the catalytic domain is the C-terminal half of the molecule, the N-terminal portion is the regulatory domain. Hexokinase V shares a similar structure. The C-terminal catalytic domain starts at approximately amino acid position 469 and continues to the end of the polypeptide sequence.

Tissue distribution of hexokinase V was also examined by Northern blot, RT-PCR and by generating specific antisera for immunologic detection in tissue sections.

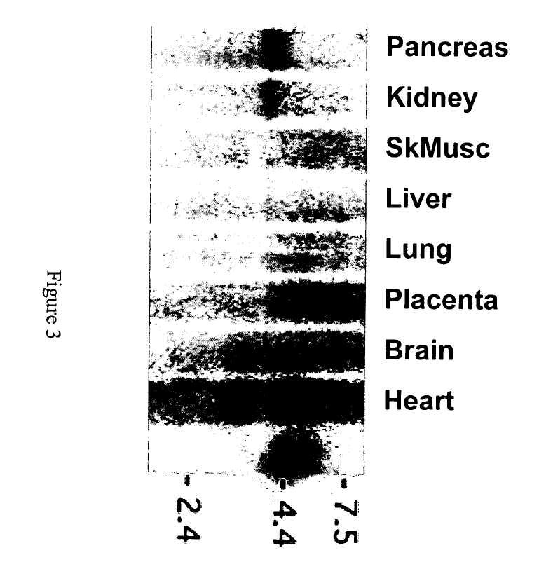

A human multiple tissue blot of human mRNAs (Clontech MTN blot, cat # 87760-1) was hybridized with an α³²P-dCTP-labeled hexokinase V probe that hybridized to the 3′ untranslated region of hexokinase V mRNA. A single band corresponding to HKV was detected in human pancreas and kidney. No hexokinase 5 transcripts were detected in the other tissues evaluated (FIG. 3).

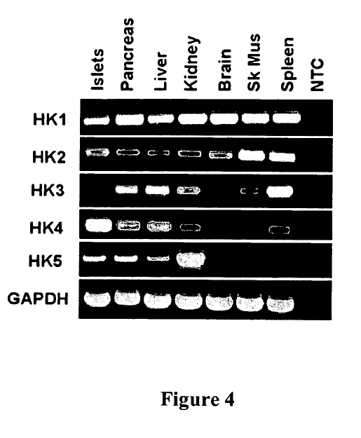

RT-PCR was also used to survey tissues for the presence or absence of each of the mammalian hexokinases (FIG. 4). Human pancreas, liver, kidney, brain, skeletal muscle and spleen cDNA were obtained from Ambion (PCR-ready cDNAs). Human islet cDNA were generated in house. For each member of the hexokinase family, gene-specific primers were designed based on the sequences. All reactions were run in parallel with GAPDH primers used as a positive control. Significant amounts of HKV mRNA were detected in human isolated pancreatic islets, human pancreas and human kidney. A faint signal was observed in human liver. Overall, these results show that HKV tissue distribution is more restricted than HKI, HKII or HKIII.

PCR primers for mammalian hexokinases tissue distribution:

| | |
|---|---|
| HKI forward | 5′GCTGGAGATGGAAAATCACACCACC3′ |
| HKI reverse | 5′CCCCCCACGAGACAAACAGAATG3′ |
| HKII forward | 5′GGGAAGGGGGAGTTTTTAGTTTGTTTTAC3′ |
| HKII reverse | 5′CCACAGGCGAATGAGGTATTTCTATGAC3′ |
| HKIII forward | 5′-TTGCGGCAGGGGGAAGAAAC-3′ |
| HKIII reverse | 5′-CACCACGAAGTCTCCTTGCTCAGTG-3′ |
| HKIV forward | 5′CTGAGTGGCTTGTGATTCTGGGATG3′ |
| HKIV reverse | 5′CTGCTTGGGGTTTCTTCCTGAGC3′ |
| HKV forward | 5′CTATGGCTTTCAGTCTTGTGGCTGC3′ |
| HKV reverse | 5′AGTGCTCCCTGGCAATCAACCTC3′ |
| Human GAPDH | 5′-GAGAAGGCTGGGGCTCATTTGC-3′ |
| forward | |
| Human GAPDH. | 5′-TGTCGCTGTTGAAGTCAGAGGAGACC-3′ |
| reverse | |

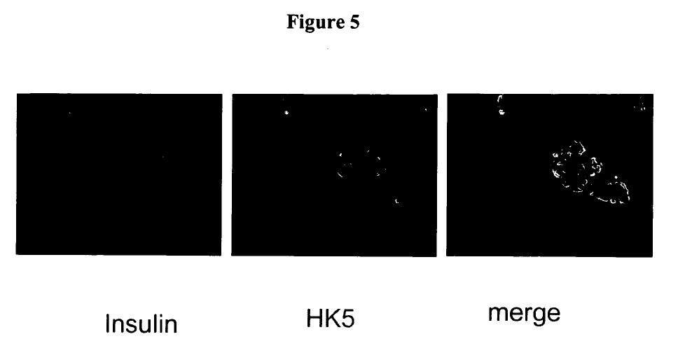

To determine the cell types within the pancreas that contain the HK V protein, we generated an affinity purified antibody to the C-terminal peptide of human HK V and performed immunofluorescent staining to localize the enzyme using human pancreas sections. Co-staining with insulin shows that within the pancreas, HK V protein is concentrated primarily in islets, but is present in the cytoplasm of both beta cells and non-beta cells within the islet (FIG. 5).

### Example 2

### Hexokinase V is Overexpressed in Diabetes

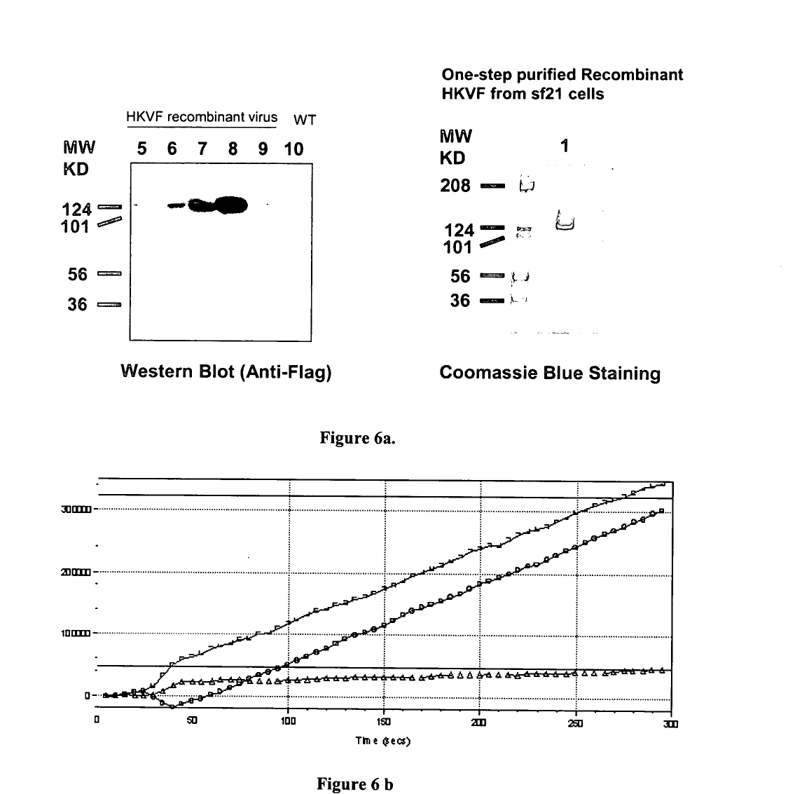

To determine whether HK V has Km for glucose that is similar to that of HK I and II (0.1-0.2 mM) or closer to that of glucokinase (8 mM), an, active recombinant version of HK V that was expressed and purified from E. coli was obtained (FIG. 6). Hexokinase activity was measured in a system coupled with glucose 6-phosphate dehydrogenase (G6PD) at 30° C. The assay mixture contained 0.1M triethanolamine, pH7.6, 6.5 mM MgCl₂, 2.7 mM ATP, 0.83 mM NADP⁺, and 0.7 U/ml G6PD in the final volume of 250 μl. The assay was performed in a 96-well plate. The enzyme was diluted in water for a final volume of 75 μl. The assay mixture was added to the enzyme, and D-glucose (5 mM final concentration) was added to the wells by a FlexStation instrument. Fluorescence was monitored over 300 seconds (5 second intervals) on the FlexStation by the reduction of NADP⁺. FlexStation settings were 350 nm excitation, 460 nm emission, and 420 nm cutoff. For each molecule of glucose 6-phosphate produced, a molecule of NADP⁺ was reduced. One unit of hexokinase activity is defined as the formation of 1 μmol of glucose 6-phosphate or ADP/minute at 30° C. Kinetic studies require dialysis or alternative methods to remove glucose from the purified HKV fractions.

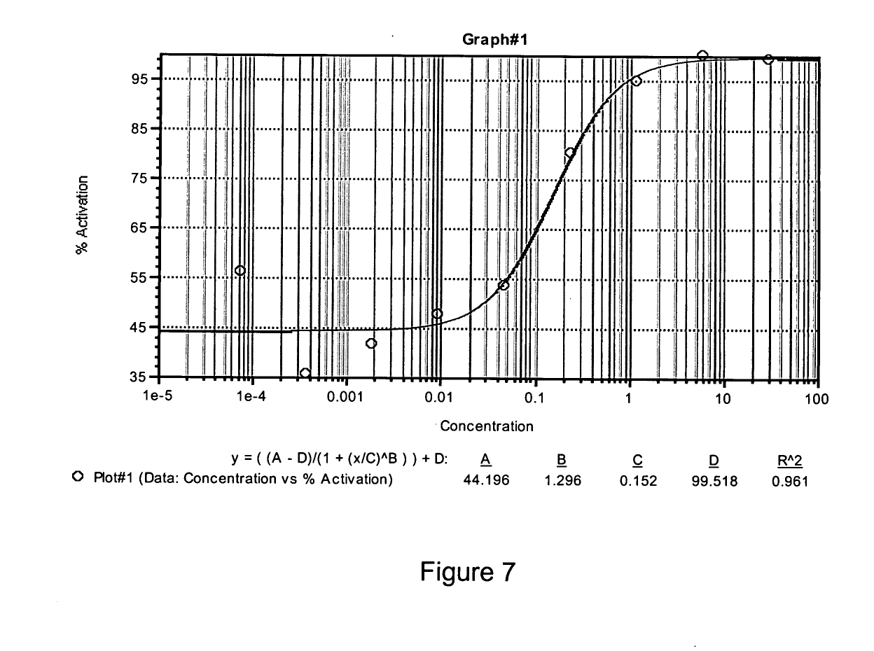

The Km of glucose for purified HK V was calculated to be 0.15 mM, which is well below the physiologically normal range for blood glucose (FIG. 7). Based on these calculations, HK V will be almost saturated with substrate even at low blood glucose concentrations.

Hexokinase V mRNA levels were evaluated by real-time PCR in islets derived from 12 week-old lean (db/+) and diabetic (db/db) mice and in islets derived from C57BL/6J mice fed a chow or high fat (58% fat) diet for 16 weeks. Significant increases in HKV mRNA levels were detected in diabetic and high-fat fed mice. The analyses were performed as follows:

The mRNA levels of mouse hexokinase V were quantified by real-time PCR using the SYBR® green I dye (SYBR® green RT-PCR reagents kit, Applied Biosystems, Foster City, Calif.). For each islet sample, 1 μg of total RNA was reverse transcribed to generate cDNA according to the manufacturer's instructions. The sequences of the gene-specific primers were as follow: mHK5: 5′-CTGCAGGAGACGGTGAAAGAG-3′ (sense) and 5′-CGCTGCCGTCTTCTGACA-3′ (antisense). Direct detection of PCR product was monitored by measuring the increase in fluorescence caused by the binding of SYBR® green dye to double stranded DNA with an ABI Prism 7700 sequence detection system (Applied Biosystems, Foster City, Calif.). Absence of non-specific or genomic amplification was assessed by including a non-template control and minus RT controls. The fluorescent signal in each sample was normalized to a corresponding β-actin signal (endogenous control) using the following mouse β-actin primers: 5′-CGTGAAAAGATGACCCAGATCA-3′ (sense) and 5′-CACAGCCTGGATGGCTACGT-3′ (antisense). Fold changes were calculated by using the comparative CT method. Results represent 2 (high fat fed) to 3 (db/db) individual islet preparations.

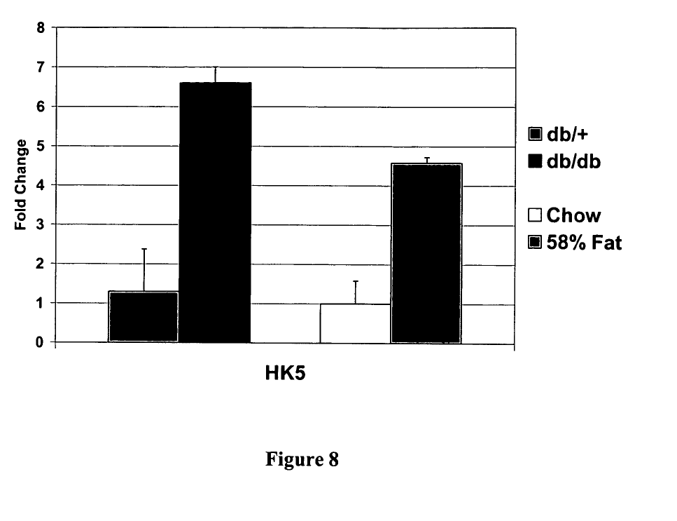

Reimer & Ahren (Diabetes February; 51 Suppl 1:S138-43, 2002) have shown that C57BL6 mice are hyperinsulinemic after 8 weeks on a high fat diet and that their isolated islets hypersecrete insulin under conditions of low (3.3 mM) glucose. In the instant example, C57BLB6 mice on the same high fat diet for 16 weeks displayed a 4-fold increase in HK V mRNA relative to mice on a chow diet (FIG. 8). The high fat fed mice are hyperinsulinemic after 5 hours fasting and display little increase in insulin in response to an IP glucose challenge (data not shown). It was also found that diabetic (db/db) mice have higher levels (5-fold) of HK V mRNA than non-diabetic (db/+) littermates (FIG. 8).

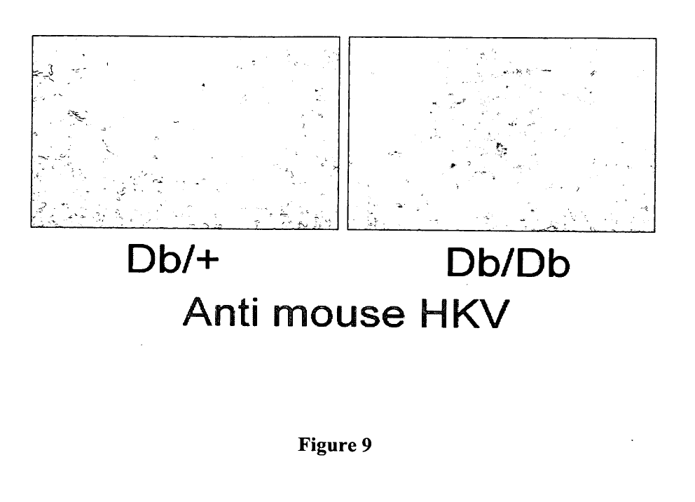

Immunohistochemical detection with an antiserum to the C-terminal peptide of mouse HK V reveals that diabetic (db/db) mouse islets also display substantially more HK V protein than non-diabetic (db/+) mouse islets (FIG. 9). The results suggest that the increased expression of HK V in high fat fed and db/db islets may contribute to the partial uncoupling of serum insulin levels from serum glucose levels that occurs in these mice.

### Example 3

### Overexpression of HKV in Islet Cells Disrupts Glucose-Stimulated Insulin Secretion

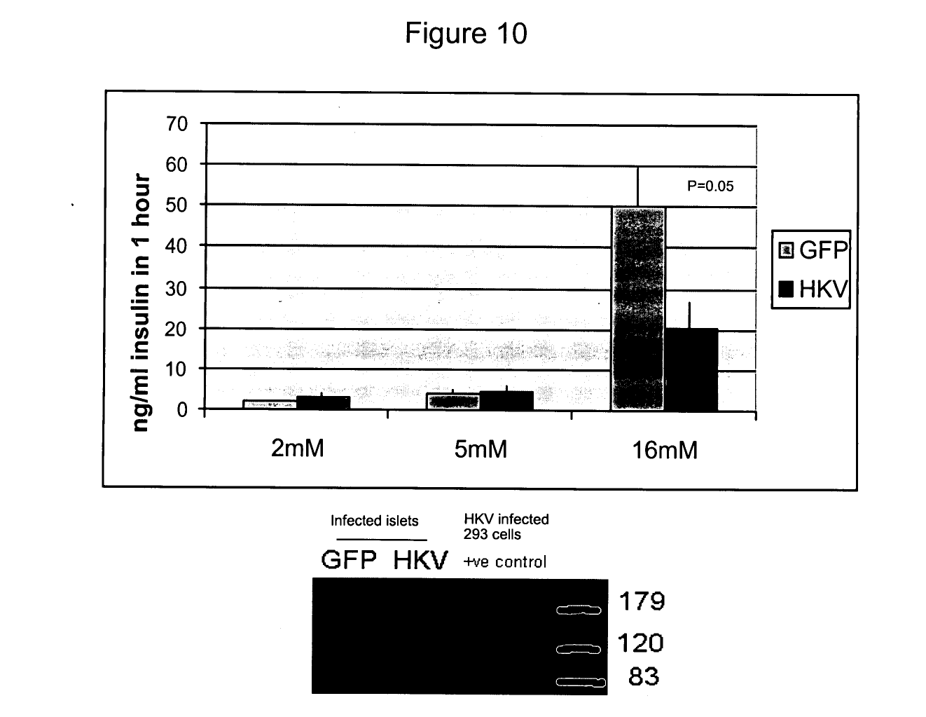

The effects of overexpression HKV in islets was examined using an adenovirus vector. Isolated rat islets were infected with 10×10⁶ pfu of adenovirus expressing either GFP or GFP and Hexokinase V. Forty eight hours after infection, the islets were pre-incubated in 2 mM glucose in KRB media, then insulin secretion was measured in response to 2, 5 and 15 mM glucose over one hour. Insulin in the media was measured by ELISA (Linco). The results shows that overexpression of hexokinase V leads to a reduction in the sensitivity of insulin secretion in response to glucose (FIG. 10).

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.one of ordinary skill in the art in light of the teachings of this invention that certain changes and modifications may be made thereto without departing from the spirit or scope of the appended claims.

All publications and patent applications cited in this specification are herein incorporated by reference as if each individual publication or patent application were specifically and individually indicated to be incorporated by reference.

Table of Sequences

SEQ ID NO:1 Human hexokinase nucleic acid sequence (start methionine
is underlined and indicated in bold; stop codon is underlined)
ATGTTTGCGGTCCACTTGATGGCATTTTACTTCAGCAAGCTGAAGGAGGACCAGATCAAGAAGGTGGA
CAGGTTCCTGTATCACATGCGGCTCTCCGATGACACCCTTTTGGACATCATGAGGCGGTTCCGGGCTG
AGATGGAGAAGGGCCTGGCAAAGGACACCAACCCCACGGCTGCAGTGAAGATGTTGCCCACCTTCGTC
AGGGCCATTCCCGATGGTTCCGAAAATGGGGAGTTCCTTTCCCTGGATCTCGGAGGGTCCAAGTTCCG
AGTGCTGAAGGTGCAAGTCGCTGAAGAGGGGAAGCGACACGTGCAGATGGAGAGTCAGTTCTACCCAA
CGCCCAATGAAATCATCCGCGGGAACGGCATAGAGCTGTTTGAATATGTAGCTGACTGTCTGGCAGAT
TTCATGAAGACCAAAGATTTAAAGCATAAGAAATTGCCCCTTGGCCTAACTTTTTCTTTCCCCTGTCG
ACAGACTAAACTGGAAGAGGGTGTCCTACTTTCGTGGACAAAAAAGTTTAAGGCACGAGGAGTTCAGG
ACACGGATGTGGTGAGCCGTCTGACCAAAGCCATGAGAAGACACAAGGACATGGACGTGGACATCCTG
GCCCTGGTCAATGACACCGTGGGGACCATGATGACCTGTGCCTATGACGACCCCTACTGCGAAGTTGG
TGTCATCATCGGAACTGGCACCAATGCGTGTTACATGGAGGACATGAGCAACATTGACCTGCTGGAGG
GCGACGAGGGCAGGATGTGCATCAACACAGAGTGGGGGGCCTTCGGGGACGACGGGGCCCTGGAGGAC
ATTCGCACTGAGTTCGACAGGGAGCTGGACCTCGGCTCTCTCAACCCAGGAAAGCAACTGTTCGAGAA
GATGATCAGTGGCCTGTACCTGGGGGAGCTTGTCAGGCTTATCTTGCTGAAGATGGCCAAGGCTGGCC
TCCTGTTTGGTGGTGAGAAATCTTCTGCTCTCCACACTAAGGGCAAGATCGAAACACGGCACGTGGCT
GCCATGGAGAAGTATAAAGAAGGCCTTGCTAATACAAGAGAGATCCTGGTGGACCTGGGTCTGGAACC
GTCTGAGGCTGACTGCATTGCCGTCCAGCATGTCTGTACCATCGTCTCCTTCCGCTCGGCCAATCTCT
GTGCAGCAGCTCTGGCGGCCATCCTGACACGCCTCCGGGAGAACAAGAAGGTGGAACGGCTCCGGACC
ACAGTGGGCATGGACGGCACCCTCTACAAGATACACCCTCAGTACCCAAAACGCCTGCACAAGGTGGT
GAGGAAACTGGTCCCAAGCTGTGATGTCCGCTTCCTCCTGTCAGAGAGTGGCAGCACCAAGGGGGCCG
CCATGGTGACCGCGGTGGCCTCCCGCGTGCAGGCCCAGCGGAAGCAGATCGACAGGGTGCTGGCTTTG
TTCCAGCTGACCCGAGAGCAGCTCGTGGACGTGCAGGCCAAGATGCGGGCTGAGCTGGAGTATGGGCT
GAAGAAGAAGAGCCACGGGCTGGCCACGGTCAGGATGCTGCCCACCTACGTCTGCGGGCTGCCGGACG
GCACAGAGAAAGGAAAGTTTCTCGCCCTGGATCTTGGGGGAACCAACTTCCGGGTCCTCCTGGTGAAG
ATCAGAAGTGGACGGAGGTCAGTGCGAATGTACAACAAGATCTTCGCCATCCCCCTGGAGATCATGCA
GGGCACTGGTGAGGAGCTCTTTGATCACATTGTGCAGTGCATCGCCGACTTCCTGGACTACATGGGCC
TCAAGGGAGCCTCCCTACCTTTGGGCTTCACATTCTCATTTCCCTGCAGGCAGATGAGCATTGACAAG
GGAACACTCATAGGGTGGACCAAAGGTTTCAAGGCCACTGACTGTGAAGGGGAGGACGTGGTGGACAT
GCTCAGGGAAGCCATCAAGAGGAGAAACGAGTTTGACCTGGACATTGTTGCAGTCGTGAATGATACAG
TGGGGACCATGATGACCTGTGGCTATGAAGATCCTAATTGTGAGATTGGCCTGATTGCAGGAACAGGC
AGCAACATGTGCTACATGGAGGACATGAGGAACATCGAGATGGTGGAGGGGGGTGAAGGGAAGATGTG
CATCAATACAGAGTGGGGAGGATTTGGAGACAATGGCTGCATAGATGACATCCGGACCCGATACGACA
CGGAGGTGGATGAGGGGTCCTTGAATCCTGGCAAGCAGAGATACGAGAAAATGACCAGTGGGATGTAC
TTGGGGGAGATTGTGCGGCAGATCCTGATCGACCTGACCAAGCAGGGTCTCCTCTTCCGAGGGCAGAT
TTCAGAGCGTCTCCGGACCAGGGGCATCTTCGAAACCAAGTTCCTGTCCCAGATCGAAAGCGATCGGC
TGGCCCTTCTCCAGGTCAGGAGGATTCTGCAGCAGCTGGGCCTGGACAGCACGTGTGAGGACAGCATC
GTGGTGAAGGAGGTGTGCGGAGCCGTGTCCCGGCGGGCGGCCCAGCTCTGCGGTGCTGGCCTGGCCGC
TATAGTGGAAAAAAGGAGAGAAGACCAGGGGCTAGAGCACCTGAGGATCACTGTGGGTGTGGACGGCA
CCCTGTACAAGCTGCACCCTCACTTTTCTAGAATATTGCAGGAAACTGTGAAGGAACTAGCCCCTCGA
TGTGATGTGACATTCATGCTGTCAGAAGATGGCAGTGGAAAAGGGGCAGCACTGATCACTGCTGTGGC
CAAGAGGTTACAGCAGGCACAGAAGGAGAACTAG
SEQ ID NO:2 Human hexokinase V amino acid sequence
MFAVHLMAFYFSKLKEDQIKKVDRFLYHMRLSDDTLLDIMRRFRAEMEKGLAKDTNPTAAVKMLPTFV
RAIPDGSENGEFLSLDLGGSKFRVLKVQVAEEGKRHVQMESQFYPTPNEIIRGNGIELFEYVADCLAD
FMKTKDLKHKKLPLGLTFSFPCRQTKLEEGVLLSWTKKFKARGVQDTDVVSRLTKAMRRHKDMDVDIL
ALVNDTVGTMMTCAYDDPYCEVGVIIGTGTNACYMEDMSNIDLVEGDEGRMCINTEWGAFGDDGALED
IRTEFDRELDLGSLNPGKQLFEKMISGLYLGELVRLILLKMAKAGLLFGGEKSSALHTKGKIETRHVA
AMEKYKEGLANTREILVDLGLEPSEADCIAVQHVCTIVSFRSANLCAAALAAILTRLRENKKVERLRT
TVGMDGTLYKIHPQYPKRLHKVVRKLVPSCDVRFLLSESGSTKGAAMVTAVASRVQAQRKQIDRVLAL
FQLTREQLVDVQAKMRAELEYGLKKKSHGLATVRMLPTYVCGLPDGTEKGKFLALDLGGTNFRVLLVK
IRSGRRSVRMYNKIFAIPLEIMQGTGEELFDHIVQCIADFLDYMGLKGASLPLGFTFSFPCRQMSIDK
GTLIGWTKGFKATDCEGEDVVDMLREAIKRRNEFDLDIVAVVNDTVGTMMTCGYEDPNCEIGLIAGTG
SNMCYMEDMRNIEMVEGGEGKMCINTEWGGFGDNGCIDDIRTRYDTEVDEGSLNPGKQRYEKMTSGMY
LGEIVRQILIDLTKQGLLFRGQISERLRTRGIFETKFLSQIESDRLALLQVRRILQQLGLDSTCEDSI
VVKEVCGAVSRRAAQLCGAGLAAIVEKRREDQGLEHLRITVGVDGTLYKLHPHFSRILQETVKELAPR
CDVTFMLSEDGSGKGAALITAVAKRLQQAQKEN
SEQ ID NO:3 variant hexokinase V nucleic acid sequence: cytosine
substitution relative to SEQ ID NO:1 at position encoding amino acid
residue at position 124 of SEQ ID NO:4 (Cytosine residue is indi-
cated in bold in large font).
CTACCATCCAGAGCCTCCTATTAGACAATCAAGTGTGTGCCAGAGGGAGGGACCAAAGGGGTGGGGTG
GGGGGGAGTTTAATCATTGAACCAAGCAGGCTGGAGGTATTTAGTCCGCAACACCTCGCTCCCCAGGA
GGTCTGCCAGCCTGGACTGGAAGCGTGCAACACTCCAGAGTCGTAGGAGTGAACACTGCACAGGAATT
CTCTGCCCATCTCAGGAGAAACCAAACTTGGGGAAAATGTTTGCGGTCCACTTGATGGCATTTTACTT
CAGCAAGCTGAAGGAGGACCAGATCAAGAAGGTGGACAGGTTCCTGTATCACATGCGGCTCTCCGATG
ACACCCTTTTGGACATCATGAGGCGGTTCCGGGCTGAGATGGAGAAGGGCCTGGCAAAGGACACCAAC
CCCACGGCTGCAGTGAAGATGTTGCCCACCTTCGTCAGGGCCATTCCCGATGGTTCCGAAAATGGGGA
GTTCCTTTCCCTGGATCTCGGAGGGTCCAAGTTCCGAGTGCTGAAGGTGCAAGTCGCTGAAGAGGGGA
AGCGACACGTGCAGATGGAGAGTCAGTTCTACCCAACGCCCAATGAAATCATCCGCGGGAACGGCACA
GAGCTGTTTGAATATGTAGCTGACTGTCTGGCAGATTTCATGAAGACCAAAGATTTAAAGCATAAGAA
ATTGCCCCTTGGCCTAACTTTTTCTTTCCCCTGTCGACAGACTAAACTGGAAGAGGGTGTCCTACTTT
CGTGGACAAAAAAGTTTAAGGCACGAGGAGTTCAGGACACGGATGTGGTGAGCCGTCTGACCAAAGCC
ATGAGAAGACACAAGGACATGGACGTGGACATCCTGGCCCTGGTCAATGACACCGTGGGGACCATGAT
GACCTGTGCCTATGACGACCCCTACTGCGAAGTTGGTGTCATCATCGGAACTGGCACCAATGCGTGTT
ACATGGAGGACATGAGCAACATTGACCTGGTGGAGGGCGACGAGGGCAGGATGTGCATCAACACAGAG
TGGGGGGCCTTCGGGGACGACGGGGCCCTGGAGGACATTCGCACTGAGTTCGACAGGGAGCTGGACCT
CGGCTCTCTCAACCCAGGAAAGCAACTGTTCGAGAAGATGATCAGTGGCCTGTACCTGGGGGAGCTTG
TCAGGCTTATCTTGCTGAAGATGGCCAAGGCTGGCCTCCTGTTTGGTGGTGAGAAATCTTCTGCTCTC
CACACTAAGGGCAAGATCGAAACACGGCACGTGGCTGCCATGGAGAAGTATAAAGAAGGCCTTGCTAA
TACAAGAGAGATCCTGGTGGACCTGGGTCTGGAACCGTCTGAGGCTGACTGCATTGCCGTCCAGCATG
TCTGTACCATCGTCTCCTTCCGCTCGGCCAATCTCTGTGCAGCAGCTCTGGCGGCCATCCTGACACGC
CTCCGGGAGAACAAGAAGGTGGAACGGCTCCGGACCACAGTGGGCATGGACGGCACCCTCTACAAGAT
ACACCCTCAGTACCCAAAACGCCTGCACAAGGTGGTGAGGAAACTGGTCCCAAGCTGTGATGTCCGCT
TCCTCCTGTCAGAGAGTGGCAGCACCAAGGGGGCCGCCATGGTGACCGCGGTGGCCTCCCGCGTGCAG
GCCCAGCGGAAGCAGATCGACAGGGTGCTGGCTTTGTTCCAGCTGACCCGAGAGCAGCTCGTGGACGT
GCAGGCCAAGATGCGGGCTGAGCTGGAGTATGGGCTGAAGAAGAAGAGCCACGGGCTGGCCACGGTCA
GGATGCTGCCCACCTACGTCTGCGGGCTGCCGGACGGCACAGAGAAAGGAAAGTTTCTCGCCCTGGAT
CTTGGGGGAACCAACTTCCGGGTCCTCCTGGTGAAGATCAGAAGTGGACGGAGGTCAGTGCGAATGTA
CAACAAGATCTTCGCCATCCCCCTGGAGATCATGCAGGGCACTGGTGAGGAGCTCTTTGATCACATTG
TGCAGTGCATCGCCGACTTCCTGGACTACATGGGCCTCAAGGGAGCCTCCCTACCTTTGGGCTTCACA
TTCTCATTTCCCTGCAGGCAGATGAGCATTGACAAGGGAACACTCATAGGGTGGACCAAAGGTTTCAA
GGCCACTGACTGTGAAGGGGAGGACGTGGTGGACATGCTCAGGGAAGCCATCAAGAGGAGAAACGAGT
TTGACCTGGACATTGTTGCAGTCGTGAATGATACAGTGGGGACCATGATGACCTGTGGCTATGAAGAT
CCTAATTGTGAGATTGGCCTGATTGCAGGAACAGGCAGCAACATGTGCTACATGGAGGACATGAGGAA
CATCGAGATGGTGGAGGGGGGTGAAGGGAAGATGTGCATCAATACAGAGTGGGGAGGATTTGGAGACA
ATGGCTGCATAGATGACATCCGGACCCGATACGACACGGAGGTGGATGAGGGGTCCTTGAATCCTGGC
AAGCAGAGATACGAGAAAATGACCAGTGGGATGTACTTGGGGGAGATTGTGCGGCAGATCCTGATCGA
CCTGACCAAGCAGGGTCTCCTCTTCCGAGGGCAGATTTCAGAGCGTCTCCGGACCAGGGGCATCTTCG
AAACCAAGTTCCTGTCCCAGATCGAAAGCGATCGGCTGGCCCTTCTCCAGGTCAGGAGGATTCTGCAG
CAGCTGGGCCTGGACAGCACGTGTGAGGACAGCATCGTGGTGAAGGAGGTGTGCGGAGCCGTGTCCCG
GCGGGCGGCCCAGCTCTGCGGTGCTGGCCTGGCCGCTATAGTGGAAAAAAGGAGAGAAGACCAGGGGC
TAGAGCACCTGAGGATCACTGTGGGTGTGGACGGCACCCTGTACAAGCTGCACCCTCACTTTTCTAGA
ATATTGCAGGAAACTGTGAAGGAACTAGCCCCTCGATGTGATGTGACATTCATGCTGTCAGAAGATGG
CAGTGGAAAAGGGGCAGCACTGATCACTGCTGTGGCCAAGAGGTTACAGCAGGCACAGAAGGAGAACT
AGGAACCCCTGGGATTGGACCTGATGCATCTTGGATACTGAACAGCTTTTCCTCTGGCAGATCAGTTG
GTCAGAGACCAATGGGCACCCTCCTGGCTGACCTCACCTTCTGGATGGCCGAAAGAGAACCCCAGGTT
CTCGGGTACTCTTAGTATCTTGTACTGGATTTGCAGTGACATTACATGACATCTCTATTTGGTATATT
TGGGCCAAAATGGGCCAACTTATGAAATCAAAGTGTCTGTCCTGAGAGATCCCCTTTCAACACATTGT
TCAGGTGAGGCTTGAGCTGTCAATTCTCTATGGCTTTCAGTCTTGTGGCTGCGGGACTTGGAAATATA
TAGAATCTGCCCATGTGGCTGGCAGGCTGTTTCCCCATTGGGATGCTTAAGCCATCTCTTATAGGGGA
TTGGACCCTGTACTTGTGGATGAACATTGGAGAGCAAGAGGAACTCACGTTATGAACTAGGGGGATCT
CATCTAACTTGTCCTTAACTTGCCATGTTGACTTCAAACCTGTTAAGAGAACAAAGACTTTGAAGTAT
CCAGCCCCAGGGTGCAGAGAGGTTGATTGCCAGGGAGCACTGCAGGAATCATTGCATGCTTAAAGCGA
GTTATGTCAGCACCCTGTAGGATTTTGTTCCTTATTAAGTGTGTGCCATGTGGTGGGGTGCTGTCTGG
GGCATCTGTTTTTCATTTTGCCTGTGGTTTGTGTTGCAGGTGTTGATAGTTGTTTTAAGGATTGTTAG
GTATAGGAAATCCAGTAAATTATAAAAAAATTTTGATTTTCCAATAAA
SEQ ID NO:4 variant hexokinase V amino acid sequence: threonine at
position 124 (shown in bold, large font)
MFAVHLMAFYFSKLKEDQIKKVDRFLYHMRLSDDTLLDIMRRFRAEMEKGLAKDTNPTAAVKMLPTFV
RAIPDGSENGEFLSLDLGGSKFRVLKVQVAEEGKRHVQMESQFYPTPNEIIRGNGTELFEYVADCLAD
FMKTKDLKHKKLPLGLTFSFPCRQTKLEEGVLLSWTKKFKARGVQDTDVVSRLTKAMRRHKDMDVDIL
ALVNDTVGTMMTCAYDDPYCEVGVIIGTGTNACYMEDMSNIDLVEGDEGRMCINTEWGAFGDDGALED
IRTEFDRELDLGSLNPGKQLFEKMISGLYLGELVRLILLKMAKAGLLFGGEKSSALHTKGKIETRHVA
AMEKYKEGLANTREILVDLGLEPSEADCIAVQHVCTIVSFRSANLCAAALAAILTRLRENKKVERLRT
TVGMDGTLYKIHPQYPKRLHKVVRKLVPSCDVRFLLSESGSTKGAAMVTAVASRVQAQRKQIDRVLAL
FQLTREQLVDVQAKMPAELEYGLKKKSHGLATVRMLPTYVCGLPDGTEKGKFLALDLGGTNFRVLLVK
IRSGRRSVRMYNKIFAIPLEIMQGTGEELFDHIVQCIADFLDYMGLKGASLPLGFTFSFPCRQMSIDK
GTLIGWTKGFKATDCEGEDVVDMLREAIKRRNEFDLDIVAVVNDTVGTMMTCGYEDPNCEIGLIAGTG
SNMCYMEDMRNIEMVEGGEGKMCINTEWGGFGDNGCIDDIRTRYDTEVDEGSLNPGKQRYEKMTSGMY
LGEIVRQILIDLTKQGLLFRGQISERLRTRGIFETKFLSQIESDRLALLQVRRILQQLGLDSTCEDSI
VVKEVCGAVSRRAAQLCGAGLAAIVEKRREDQGLEHLRITVGVDGTLYKLHPHFSRILQETVKELAPR
CDVTFMLSEDGSGKGAALITAVAKRLQQAQKEN

1. A method for identifying an agent for treating a diabetic or pre-diabetic individual, the method comprising the steps of:
(i) contacting a candidate agent with a kidney or pancreatic cell that expresses a nucleic acid encoding a polypeptide having glucose phosphorylating activity that comprises at least 20 contiguous amino acids of SEQ ID NO:2;
(ii) determining the activity of the polypeptide; and
(ii) selecting an agent that inhibits the activity of the polypeptide, thereby identifying an agent for treating a diabetic or pre-diabetic individual.

2. The method of claim 1, wherein the polypeptide comprises SEQ ID NO:2.

3. The method of claim 1, wherein the polypeptide is overexpressed relative to normal.

4. The method of claim 1, wherein the cell is a pancreatic cell.

5. The method of claim 4, wherein the pancreatic cell is from a diabetic animal.

6. The method of claim 1, wherein the step of determining the activity of activity of the polypeptide comprises determining the ability of the polypeptide to phosphorylate a hexose.

7. The method of claim 1, wherein the step of determining the activity of the polypeptide comprises determining the amount of protein present using an immunoassay.

8. The method of claim 1, wherein the agent is an siRNA.

9. The method of claim 1, wherein the agent is an antisense RNA.

10. A method for identifying an agent for treating a diabetic or pre-diabetic individual, the method comprising the steps of:
(i) contacting a candidate agent with a kidney or pancreatic cell that expresses a nucleic acid encoding a polypeptide having glucose phosphorylating activity that comprises at least 20 contiguous amino acids of SEQ ID NO:2;
(ii) determining the level of an RNA that encodes the polypeptide; and
(ii) selecting an agent that inhibits the activity of the polypeptide, thereby identifying an agent for treating a diabetic or pre-diabetic individual.

11. The method of claim 10, wherein the polypeptide comprises SEQ ID NO:2.

12. The method of claim 10, wherein the cell is a pancreatic cell.

13. The method of claim 10 wherein the pancreatic cell is from a diabetic animal.

14. The method of claim 10, wherein the step of determining the level of an RNA that encodes the polypeptide comprises an amplification reaction.

15. The method of claim 10, wherein the cell is a pancreatic islet cell.

16. The method of claim 10, wherein the agent is an siRNA.

17. The method of claim 10, wherein the agent is an antisense RNA.

18. The method of claim 1 or claim 10, further comprising:
administering the agent to a diabetic or pre-diabetic animal;
determining the response of the animal to glucose; and
selecting a candidate agent that improves the response to glucose.

19. The method of claim 18, wherein the step of determining the response of the animal to glucose comprises determining the level of glucose-induced insulin secretion.

20. The method of claim 1 or claim 10, further comprising:
administering the agent to an animal that is a diabetic or pre-diabetic model;
determining the level of the polypeptide or the nucleic acid encoding the polypeptide in a pancreatic sample from the animal; and
selecting the candidate agent that decreases the level of the polypeptide or the nucleic acid.

21. A method for identifying an agent for treating a diabetic or pre-diabetic individual, the method comprising the steps of:
(i) contacting a candidate agent with a polypeptide having glucose phosphorylating activity that comprises at least 20 contiguous amino acids of SEQ ID NO:2;
(ii) determining binding of the agent to the polypeptide;
(iii) selecting an agent that binds to the polypeptide;
(iv) administering the agent to a diabetic or pre-diabetic animal;
(v) determining the response of the animal to glucose; and
(vi) selecting an agent that improves the response to glucose.

22. The method of claim 21, wherein the step of determining binding of the agent to the polypeptide comprises determining the activity of the polypeptide.

23. The method of claim 21, wherein the step of determining the response of the animal to glucose comprises determining the level of glucose-induced insulin secretion.

24. A method of regulating glucose sensitivity in a diabetic animal or a pre-diabetic animal, the method comprising administering to the animal a therapeutically effective amount of an agent identified by the method of claim 1, claim 10, or claim 24.

25. The method of claim 24, wherein the agent is administered to pancreatic tissue.

26. The method of claim 24, wherein the animal is a human.

27. A method of introducing an expression cassette into a pancreatic cell, the method comprising,
introducing into the cell an expression vector comprising a nucleic acid that, when expressed, inhibits the expression of a nucleic acid encoding a polypeptide having glucose phosphorylating activity that comprises at least 20 contiguous amino acids of SEQ ID NO:2.

28. The method of claim 27, wherein the polypeptide comprises SEQ ID NO:2.

29. The method of claim 27, further comprising introducing the cell into a diabetic animal.

30. The method of claim 29, wherein the cell is from the human.

31. A method of diagnosing a prediabetic or diabetic patient; the method comprising:
detecting an increase, relative to normal, in the level of a polypeptide of SEQ ID NO:2 in a sample from the patient, thereby diagnosing the diabetic or prediabetic patient.

32. An isolated nucleic acid encoding a polypeptide comprising an amino acid sequence as set forth in SEQ ID NO:4.

33. An isolated nucleic acid comprising the nucleic acid sequence of SEQ ID NO:3.


SEQUENCE LISTING (26 sequences)

SEQ ID NO: 1 - DNA, 2754 bp, Homo sapiens
  human hexokinase V (HKV, HK5)
atgtttgcggtccacttgatggcattttacttcagcaagctgaaggaggaccagatcaagaaggtggacaggttcctgtatcacatgcggctctccgatgacacccttttggacatcatgaggcggttccgggctgagatggagaagggcctggcaaaggacaccaaccccacggctgcagtgaagatgttgcccaccttcgtcagggccattcccgatggttccgaaaatggggagttcctttccctggatctcggagggtccaagttccgagtgctgaaggtgcaagtcgctgaagaggggaagcgacacgtgcagatggagagtcagttctacccaacgcccaatgaaatcatccgcgggaacggcatagagctgtttgaatatgtagctgactgtctggcagatttcatgaagaccaaagatttaaagcataagaaattgccccttggcctaactttttctttcccctgtcgacagactaaactggaagagggtgtcctactttcgtggacaaaaaagtttaaggcacgaggagttcaggacacggatgtggtgagccgtctgaccaaagccatgagaagacacaaggacatggacgtggacatcctggccctggtcaatgacaccgtggggaccatgatgacctgtgcctatgacgacccctactgcgaagttggtgtcatcatcggaactggcaccaatgcgtgttacatggaggacatgagcaacattgacctggtggagggcgacgagggcaggatgtgcatcaacacagagtggggggccttcggggacgacggggccctggaggacattcgcactgagttcgacagggagctggacctcggctctctcaacccaggaaagcaactgttcgagaagatgatcagtggcctgtacctgggggagcttgtcaggcttatcttgctgaagatggccaaggctggcctcctgtttggtggtgagaaatcttctgctctccacactaagggcaagatcgaaacacggcacgtggctgccatggagaagtataaagaaggccttgctaatacaagagagatcctggtggacctgggtctggaaccgtctgaggctgactgcattgccgtccagcatgtctgtaccatcgtctccttccgctcggccaatctctgtgcagcagctctggcggccatcctgacacgcctccgggagaacaagaaggtggaacggctccggaccacagtgggcatggacggcaccctctacaagatacaccctcagtacccaaaacgcctgcacaaggtggtgaggaaactggtcccaagctgtgatgtccgcttcctcctgtcagagagtggcagcaccaagggggccgccatggtgaccgcggtggcctcccgcgtgcaggcccagcggaagcagatcgacagggtgctggctttgttccagctgacccgagagcagctcgtggacgtgcaggccaagatgcgggctgagctggagtatgggctgaagaagaagagccacgggctggccacggtcaggatgctgcccacctacgtctgcgggctgccggacggcacagagaaaggaaagtttctcgccctggatcttgggggaaccaacttccgggtcctcctggtgaagatcagaagtggacggaggtcagtgcgaatgtacaacaagatcttcgccatccccctggagatcatgcagggcactggtgaggagctctttgatcacattgtgcagtgcatcgccgacttcctggactacatgggcctcaagggagcctccctacctttgggcttcacattctcatttccctgcaggcagatgagcattgacaagggaacactcatagggtggaccaaaggtttcaaggccactgactgtgaaggggaggacgtggtggacatgctcagggaagccatcaagaggagaaacgagtttgacctggacattgttgcagtcgtgaatgatacagtggggaccatgatgacctgtggctatgaagatcctaattgtgagattggcctgattgcaggaacaggcagcaacatgtgctacatggaggacatgaggaacatcgagatggtggaggggggtgaagggaagatgtgcatcaatacagagtggggaggatttggagacaatggctgcatagatgacatccggacccgatacgacacggaggtggatgaggggtccttgaatcctggcaagcagagatacgagaaaatgaccagtgggatgtacttgggggagattgtgcggcagatcctgatcgacctgaccaagcagggtctcctcttccgagggcagatttcagagcgtctccggaccaggggcatcttcgaaaccaagttcctgtcccagatcgaaagcgatcggctggcccttctccaggtcaggaggattctgcagcagctgggcctggacagcacgtgtgaggacagcatcgtggtgaaggaggtgtgcggagccgtgtcccggcgggcggcccagctctgcggtgctggcctggccgctatagtggaaaaaaggagagaagaccaggggctagagcacctgaggatcactgtgggtgtggacggcaccctgtacaagctgcaccctcacttttctagaatattgcaggaaactgtgaaggaactagcccctcgatgtgatgtgacattcatgctgtcagaagatggcagtggaaaaggggcagcactgatcactgctgtggccaagaggttacagcaggcacagaaggagaactag

SEQ ID NO: 2 - protein, 917 aa, Homo sapiens [sequence omitted]

SEQ ID NO: 3 - DNA, 3788 bp, Homo sapiens
  human hexokinase V variant
ctaccatccagagcctcctattagacaatcaagtgtgtgccagagggagggaccaaaggggtggggtgggggggagtttaatcattgaaccaagcaggctggaggtatttagtccgcaacacctcgctccccaggaggtctgccagcctggactggaagcgtgcaacactccagagtcgtaggagtgaacactgcacaggaattctctgcccatctcaggagaaaccaaacttggggaaaatgtttgcggtccacttgatggcattttacttcagcaagctgaaggaggaccagatcaagaaggtggacaggttcctgtatcacatgcggctctccgatgacacccttttggacatcatgaggcggttccgggctgagatggagaagggcctggcaaaggacaccaaccccacggctgcagtgaagatgttgcccaccttcgtcagggccattcccgatggttccgaaaatggggagttcctttccctggatctcggagggtccaagttccgagtgctgaaggtgcaagtcgctgaagaggggaagcgacacgtgcagatggagagtcagttctacccaacgcccaatgaaatcatccgcgggaacggcacagagctgtttgaatatgtagctgactgtctggcagatttcatgaagaccaaagatttaaagcataagaaattgccccttggcctaactttttctttcccctgtcgacagactaaactggaagagggtgtcctactttcgtggacaaaaaagtttaaggcacgaggagttcaggacacggatgtggtgagccgtctgaccaaagccatgagaagacacaaggacatggacgtggacatcctggccctggtcaatgacaccgtggggaccatgatgacctgtgcctatgacgacccctactgcgaagttggtgtcatcatcggaactggcaccaatgcgtgttacatggaggacatgagcaacattgacctggtggagggcgacgagggcaggatgtgcatcaacacagagtggggggccttcggggacgacggggccctggaggacattcgcactgagttcgacagggagctggacctcggctctctcaacccaggaaagcaactgttcgagaagatgatcagtggcctgtacctgggggagcttgtcaggcttatcttgctgaagatggccaaggctggcctcctgtttggtggtgagaaatcttctgctctccacactaagggcaagatcgaaacacggcacgtggctgccatggagaagtataaagaaggccttgctaatacaagagagatcctggtggacctgggtctggaaccgtctgaggctgactgcattgccgtccagcatgtctgtaccatcgtctccttccgctcggccaatctctgtgcagcagctctggcggccatcctgacacgcctccgggagaacaagaaggtggaacggctccggaccacagtgggcatggacggcaccctctacaagatacaccctcagtacccaaaacgcctgcacaaggtggtgaggaaactggtcccaagctgtgatgtccgcttcctcctgtcagagagtggcagcaccaagggggccgccatggtgaccgcggtggcctcccgcgtgcaggcccagcggaagcagatcgacagggtgctggctttgttccagctgacccgagagcagctcgtggacgtgcaggccaagatgcgggctgagctggagtatgggctgaagaagaagagccacgggctggccacggtcaggatgctgcccacctacgtctgcgggctgccggacggcacagagaaaggaaagtttctcgccctggatcttgggggaaccaacttccgggtcctcctggtgaagatcagaagtggacggaggtcagtgcgaatgtacaacaagatcttcgccatccccctggagatcatgcagggcactggtgaggagctctttgatcacattgtgcagtgcatcgccgacttcctggactacatgggcctcaagggagcctccctacctttgggcttcacattctcatttccctgcaggcagatgagcattgacaagggaacactcatagggtggaccaaaggtttcaaggccactgactgtgaaggggaggacgtggtggacatgctcagggaagccatcaagaggagaaacgagtttgacctggacattgttgcagtcgtgaatgatacagtggggaccatgatgacctgtggctatgaagatcctaattgtgagattggcctgattgcaggaacaggcagcaacatgtgctacatggaggacatgaggaacatcgagatggtggaggggggtgaagggaagatgtgcatcaatacagagtggggaggatttggagacaatggctgcatagatgacatccggacccgatacgacacggaggtggatgaggggtccttgaatcctggcaagcagagatacgagaaaatgaccagtgggatgtacttgggggagattgtgcggcagatcctgatcgacctgaccaagcagggtctcctcttccgagggcagatttcagagcgtctccggaccaggggcatcttcgaaaccaagttcctgtcccagatcgaaagcgatcggctggcccttctccaggtcaggaggattctgcagcagctgggcctggacagcacgtgtgaggacagcatcgtggtgaaggaggtgtgcggagccgtgtcccggcgggcggcccagctctgcggtgctggcctggccgctatagtggaaaaaaggagagaagaccaggggctagagcacctgaggatcactgtgggtgtggacggcaccctgtacaagctgcaccctcacttttctagaatattgcaggaaactgtgaaggaactagcccctcgatgtgatgtgacattcatgctgtcagaagatggcagtggaaaaggggcagcactgatcactgctgtggccaagaggttacagcaggcacagaaggagaactaggaacccctgggattggacctgatgcatcttggatactgaacagcttttcctctggcagatcagttggtcagagaccaatgggcaccctcctggctgacctcaccttctggatggccgaaagagaaccccaggttctcgggtactcttagtatcttgtactggatttgcagtgacattacatgacatctctatttggtatatttgggccaaaatgggccaacttatgaaatcaaagtgtctgtcctgagagatcccctttcaacacattgttcaggtgaggcttgagctgtcaattctctatggctttcagtcttgtggctgcgggacttggaaatatatagaatctgcccatgtggctggcaggctgtttccccattgggatgcttaagccatctcttataggggattggaccctgtacttgtggatgaacattggagagcaagaggaactcacgttatgaactagggggatctcatctaacttgtccttaacttgccatgttgacttcaaacctgttaagagaacaaagactttgaagtatccagccccagggtgcagagaggttgattgccagggagcactgcaggaatcattgcatgcttaaagcgagttatgtcagcaccctgtaggattttgttccttattaagtgtgtgccatgtggtggggtgctgtctggggcatctgtttttcattttgcctgtggtttgtgttgcaggtgttgatagttgttttaaggattgttaggtataggaaatccagtaaattataaaaaaattttgattttccaataaa

SEQ ID NO: 4 - protein, 917 aa, Homo sapiens
  human hexokinase V variant
MFAVHLMAFYFSKLKEDQIKKVDRFLYHMRLSDDTLLDIMRRFRAEMEKGLAKDTNPTAAVKMLPTFVRAIPDGSENGEFLSLDLGGSKFRVLKVQVAEEGKRHVQMESQFYPTPNEIIRGNGTELFEYVADCLADFMKTKDLKHKKLPLGLTFSFPCRQTKLEEGVLLSWTKKFKARGVQDTDVVSRLTKAMRRHKDMDVDILALVNDTVGTMMTCAYDDPYCEVGVIIGTGTNACYMEDMSNIDLVEGDEGRMCINTEWGAFGDDGALEDIRTEFDRELDLGSLNPGKQLFEKMISGLYLGELVRLILLKMAKAGLLFGGEKSSALHTKGKIETRHVAAMEKYKEGLANTREILVDLGLEPSEADCIAVQHVCTIVSFRSANLCAAALAAILTRLRENKKVERLRTTVGMDGTLYKIHPQYPKRLHKVVRKLVPSCDVRFLLSESGSTKGAAMVTAVASRVQAQRKQIDRVLALFQLTREQLVDVQAKMRAELEYGLKKKSHGLATVRMLPTYVCGLPDGTEKGKFLALDLGGTNFRVLLVKIRSGRRSVRMYNKIFAIPLEIMQGTGEELFDHIVQCIADFLDYMGLKGASLPLGFTFSFPCRQMSIDKGTLIGWTKGFKATDCEGEDVVDMLREAIKRRNEFDLDIVAVVNDTVGTMMTCGYEDPNCEIGLIAGTGSNMCYMEDMRNIEMVEGGEGKMCINTEWGGFGDNGCIDDIRTRYDTEVDEGSLNPGKQRYEKMTSGMYLGEIVRQILIDLTKQGLLFRGQISERLRTRGIFETKFLSQIESDRLALLQVRRILQQLGLDSTCEDSIVVKEVCGAVSRRAAQLCGAGLAAIVEKRREDQGLEHLRITVGVDGTLYKLHPHFSRILQETVKELAPRCDVTFMLSEDGSGKGAALITAVAKRLQQAQKEN

SEQ ID NO: 5 - protein, 917 aa, Homo sapiens
  human hexokinase I (HK1)
MIAAQLLAYYFTELKDDQVKKIDKYLYAMRLSDETLIDIMTRFRKEMKNGLSRDFNPTATVKMLPTFVRSIPDGSEKGDFIALDLGGSSFRILRVQVNHEKNQNVHMESEVYDTPENIVHGSGSQLFDHVAECLGDFMEKRKIKDKKLPVGFTFSFPCQQSKIDEAILITWTKRFKASGVEGADVVKLLNKAIKKRGDYDANIVAVVNDTVGTMMTCGYDDQHCEVGLIIGTGTNACYMEELRHIDLVEGDEGRMCINTEWGAFGDDGSLEDIRTEFDREIDRGSLNPGKQLFEKMVSGMYLGELVRLILVKMAKEGLLFEGRITPELLTRGKFNTSDVSAIEKNKEGLHNAKEILTRLGVEPSDDDCVSVQHVCTIVSFRSANLVAATLGAILNRLRDNKGTPRLRTTVGVDGSLYKTHPQYSRRFHKTLRRLVPDSDVRFLLSESGSGKGAAMVTAVAYRLAEQHRQIEETLAHFHLTKDMLLEVKKRMRAEMELGLRKQTHNNAVVKMLPSFVRRTPDGTENGDFLALDLGGTNFRVLLVKIRSGKKRTVEMHNKIYAIPIEIMQGTGEELFDHIVSCISDFLDYMGIKGPRMPLGFTFSFPCQQTSLDAGILITWTKGFKATDCVGHDVVTLLRDAIKRREEFDLDVVAVVNDTVGTMMTCAYEEPTCEVGLIVGTGSNACYMEEMKNVEMVEGDQGQMCINMEWGAFGDNGCLDDIRTHYDRLVNEYSLNAGKQRYEKMISGMYLGEIVRNILIDFTKKGFLFRGQISETMKTRGIFETKFLSQIESDRLALLQVRAILQQLGLNSTCDDSILVKTVCGVVSRRAAQLCGAGMAAVVDKIRENRGLDRLNVTVGVDGTLYKLHPHFSRIMHQTVKELSPKCNVSFLLSEDGSGKGAALITAVGVRLRTEASS

SEQ ID NO: 6 - protein, 917 aa, Homo sapiens
  human hexokinase II (HK2)
MIASHLLAYFFTELNHDQVQKVDQYLYHMRLSDETLLEISKRFRKEMEKGLGATTHPTAAVKMLPTFVRSTPDGTEHGEFLALDLGGTNFRVLWVKVTDNGLQKVEMENQIYAIPEDIMRGSGTQLFDHIAECLANFMDKLQIKDKKLPLGFTFSFPCHQTKLDESFLVSWTKGFKSSGVEGRDVVALIRKAIQRRGDFDIDIVAVVNDTVGTMMTCGYDDHNCEIGLIVGTGSNACYMEEMRHIDMVEGDEGRMCINMEWGAFGDDGSLNDIRTEFDQEIDMGSLNPGKQLFEKMISGMYMGELVRLILVKMAKEELLFGGKLSPELLNTGRFETKDISDIEGEKDGIRKAREVLMRLGLDPTQEDCVATHRICQIVSTRSASLCAATLAAVLQRIKENKGEERLRSTIGVDGSVYKKHPHFAKRLHKTVRRLVPGCDVRFLRSEDGSGKGAAMVTAVAYRLADQHRARQKTLEHLQLSHDQLLEVKRRMKVEMERGLSKETHASAPVKMLPTYVCATPDGTEKGDFLALDLGGTNFRVLLVRVRNGKWGGVEMHNKIYAIPQEVMHGTGDELFDHIVQCIADFLEYMGMKGVSLPLGFTFSFPCQQNSLDESILLKWTKGFKASGCEGEDVVTLLKEAIHRREEFDLDVVAVVNDTVGTMMTCGFEDPHCEVGLIVGTGSNACYMEEMRNVELVEGEEGRMCVNMEWGAFGDNGCLDDFRTEFDVAVDELSLNPGKQRFEKMISGMYLGEIVRNILIDFTKRGLLFRGRISERLKTRGIFETKFLSQIESDCLALLQVRAILQHLGLESTCDDSIIVKEVCTVVARRAAQLCGAGMAAVVDRIRENRGLDALKVTVGVDGTLYKLHPHFAKVMHETVKDLAPKCDVSFLQSEDGSGKGAALITAVACRIREAGQR

SEQ ID NO: 7 - protein, 923 aa, Homo sapiens
  human hexokinase III (HK3)
MDSIGSSGLRQGEETLSCSEEGLPGPSDSSELVQECLQQFKVTRAQLQQIQASLLGSMEQALRGQASPAPAVRMLPTYVGSTPHGTEQGDFVVLELGATGASLRVLWVTLTGIEGHRVEPRSQEFVIPQEVMLGAGQQLFDFAAHCLSEFLDAQPVNKQGLQLGFSFSFPCHQTGLDRSTLISWTKGFRCSGVEGQDVVQLLRDAIRRQGAYNIDVVAVVNDTVGTMMGCEPGVRPCEVGLVVDTGTNACYMEEARHVAVLDEDRGRVCVSVEWGSLSDDGALGPVLTTFDHTLDHESLNPGAQRFEKMIGGLYLGELVRLVLAHLARCGVLFGGCTSPALLSQGSILLEHVAEMEDPSTGAARVHAILQDLGLSPGASDVELVQHVCAAVCTRAAQLCAAALAAVLSCLQHSREQQTLQVAVATGGRVCERHPRFCSVLQGTVMLLAPECDVSLIPSVDGGGRGVAMVTAVAARLAAHRRLLEETLAPFRLNHDQLAAVQAQMRKAMAKGLRGEASSLRMLPTFVRATPDGSERGDFLALDLGGTNFRVLLVRVTTGVQITSEIYSIPETVAQGSGQQLFDHIVDCIVDFQQKQGLSGQSLPLGFTFSFPCRQLGLDQGILLNWTKGFKASDCEGQDVVSLLREAITRRQAVELNVVAIVNDTVGTMMSCGYEDPRCEIGLIVGTGTNACYMEELRNVAGVPGDSGRMCINMEWGAFGDDGSLAMLSTRFDASVDQASINPGKQRFEKMISGMYLGEIVRHILLHLTSLGVLFRGQQIQRLQTRDIFKTKFLSEIESDSLALRQVRAILEDLGLPLTSDDALMVLEVCQAVSQRAAQLCGAGVAAVVEKIRGNRGLEELAVSVGVDGTLYKLHPRFSSLVAATVRELAPRCVVTFLQSEDGSGKGAALVTAVACRLAQLTRV

SEQ ID NO: 8 - protein, 465 aa, Homo sapiens [sequence omitted]

SEQ ID NO: 9 - protein, 6 aa, Artificial Sequence
  Description of Artificial Sequence hexahistidine affinity tag
HHHHHH

SEQ ID NO: 10 - protein, 200 aa, Artificial Sequence
  Description of Artificial Sequencepoly-Gly flexible linker
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 11 - DNA, 25 bp, Artificial Sequence
  Description of Artificial SequenceRT-PCR gene-specific primer HKI forward
gctggagatggaaaatcacaccacc

SEQ ID NO: 12 - DNA, 23 bp, Artificial Sequence
  Description of Artificial SequenceRT-PCR gene-specific primer HKI reverse
ccccccacgagacaaacagaatg

SEQ ID NO: 13 - DNA, 29 bp, Artificial Sequence
  Description of Artificial SequenceRT-PCR gene-specific primer HKII forward
gggaagggggagtttttagtttgttttac

SEQ ID NO: 14 - DNA, 28 bp, Artificial Sequence
  Description of Artificial SequenceRT-PCR gene-specific primer HKII reverse
ccacaggcgaatgaggtatttctatgac

SEQ ID NO: 15 - DNA, 20 bp, Artificial Sequence
  Description of Artificial SequenceRT-PCR gene-specific primer HKIII forward
ttgcggcagggggaagaaac

SEQ ID NO: 16 - DNA, 25 bp, Artificial Sequence
  Description of Artificial SequenceRT-PCR gene-specific primer HKIII reverse
caccacgaagtctccttgctcagtg

SEQ ID NO: 17 - DNA, 25 bp, Artificial Sequence
  Description of Artificial SequenceRT-PCR gene-specific primer HKIV forward
ctgagtggcttgtgattctgggatg

SEQ ID NO: 18 - DNA, 23 bp, Artificial Sequence
  Description of Artificial SequenceRT-PCR gene-specific primer HKIV reverse
ctgcttggggtttcttcctgagc

SEQ ID NO: 19 - DNA, 25 bp, Artificial Sequence
  Description of Artificial SequenceRT-PCR gene-specific primer HKV forward
ctatggctttcagtcttgtggctgc

SEQ ID NO: 20 - DNA, 23 bp, Artificial Sequence
  Description of Artificial SequenceRT-PCR gene-specific primer HKV reverse
agtgctccctggcaatcaacctc

SEQ ID NO: 21 - DNA, 22 bp, Artificial Sequence
  Description of Artificial SequenceRT-PCR gene-specific positive control primer Human GAPDH forward
gagaaggctggggctcatttgc

SEQ ID NO: 22 - DNA, 26 bp, Artificial Sequence
  Description of Artificial SequenceRT-PCR gene-specific positive control primer Human GAPDH reverse
tgtcgctgttgaagtcagaggagacc

SEQ ID NO: 23 - DNA, 21 bp, Artificial Sequence
  Description of Artificial Sequencequantitative real-time PCR gene-specific primer mHK5 (sense)
ctgcaggagacggtgaaagag

SEQ ID NO: 24 - DNA, 18 bp, Artificial Sequence
  Description of Artificial Sequencequantitative real-time PCR gene-specific primer mHK5 (antisense)
cgctgccgtcttctgaca

SEQ ID NO: 25 - DNA, 22 bp, Artificial Sequence
  Description of Artificial Sequenceendogenous control mouse beta-actin primer (sense)
cgtgaaaagatgacccagatca

SEQ ID NO: 26 - DNA, 20 bp, Artificial Sequence
  Description of Artificial Sequenceendogenous control mouse beta-actin primer (antisense)
cacagcctggatggctacgt